(19) United States
(12) Patent Application Publication
(10) Pub. No.: US 2008/0261923 A1
(43) Pub. Date: Oct. 23, 2008

(54) Alkene Mimics

(76) Inventors: Felicia A. Etzkorn, Blacksburg, VA (US); Xiaodong X. Wang, Maricopa, AZ (US); Bulling Xu, Blacksburg, VA (US)

Correspondence Address: WHITHAM, CURTIS & CHRISTOFFERSON & COOK, P.C., 11491 SUNSET HILLS ROAD, SUITE 340, RESTON, VA, 20190, US

(21) Appl. No.: 11/573,170

(22) PCT Filed: Jul. 29, 2005
(86) PCT No.: PCT/US05/26821

§ 371 Date: Sep. 26, 2007

Related U.S. Application Data
(60) Provisional application No. 60/598,421, filed on Aug. 4, 2004.

(51) Int. Cl. A61K 31/675; C07F 9/06; A61P 35/00; A61K 31/662

(52) U.S. Cl. 514/80; 546/22; 548/414; 546/23; 548/112; 558/166; 514/89; 514/114

(57) ABSTRACT

Ac-Phe-Tyr-phosphoSer-Ψ[CH═C]-Pro-Arg-NH₂AND Fmoc-bis(pivaloylmethoxy)phosphoSer-Ψ[CH═C]-Pro-2-aminoethyl-(3-indole); and their Phospho-(D)-serine stereoisomers are novel compounds. Ψ refers to a pseudo amide. Such novel compounds advantageously may be used as alkene mimics.

### FIELD OF THE INVENTION

This invention relates to the design and synthesis of compounds that are alkene mimics.

### BACKGROUND OF THE INVENTION

Certain small molecules were designed to mimic peptides in order to determine which amide form is critical to the biological function of peptidyl-prolyl isomerases (PPIases), such as cyclophilin, with particular attention to (Z)-alkene mimics. Hart and Etzkorn (2000); Hart, Trindle and Etzkorn (2001). In about April 2002, drug design to stop the cancer cell cycle was under consideration, and the cell-cycle-regulating enzyme, Pin1, was targeted, with an eye towards anticancer activity. (Virginia Tech Press release dated Apr. 10, 2002, “Chemists Explore the Shape of the Key that Signals Cell Division in Cancer Cells). At that time, the single known inhibitor of Pin1 was a natural product, juglone, that is not specific for Pin1 and is a poor inhibitor. Hennig, L., Christner, C., Kipping, M., Schelbert, B., Rucknagel, K. P., Grabley, S., Kullertz, G., and Fischer, G. (1998), Selective inactivation of parvulin-like peptidyl-prolyl cis/trans isomerases by juglone, Biochemistry 37, 5953-5960.

Regulation of the cell cycle is of fundamental significance in developmental biology and gives rise to cancer when it goes awry. The enzyme Pin1 is a phosphorylation-dependent peptidyl-prolyl isomerase (PPIase) enzyme thought to regulate mitosis via cis-trans isomerization of phosphoSer-Pro amide bonds in a variety of cell cycle proteins. Lu, K. P., Hanes, S. D., and Hunter, T. (1996), A human peptidyl-prolyl isomerase essential for regulation of mitosis, Nature 380, 544-547; Yaffe, M. B., Schutkowski, M., Shen, M., Zhou, X. Z., Stukenberg, P. T., Rahfeld, J.-U., Xu, J., Kuang, J., Kirshcner, M. W., Fischer, G., Cantley, L. C., and Lu, K. P., Science 278 (1997) 1957. In particular, Pin1 has been shown to bind phosphoSer-Pro epitopes in cdc25 phosphatase, a key regulator of the cdc2/cyclinB complex. King, R. W., Jackson, P. K., and Kirschner, M. W., Cell 79 (1994) 563. The central role Pin1 plays in the cell cycle makes Pin1 an interesting target for inhibition, both for potential anti-cancer activity and for elucidation of the mechanism of mitosis regulation. It has been proposed that Pin1 recognition of the phosphoSer-Pro amide bond acts as a conformational switch in the cell cycle. Shen, M., Stukenberg, P. T., Kirschner, M. W., and Lu, K. P., Genes Dev. 12 (1998) 706.

Preference for phosphorylated substrates by Pin1 has been clearly demonstrated (Yaffe, supra), with the central dipeptide phosphoSer-Pro as the primary recognition element. Successful laboratory work has been accomplished using a (Z)-alkene amide bond isostere to mimic the Ala-cis-Pro amide bond for the inhibition of the PPIase cyclophilin, which then led to design of an analogous inhibitor based on a substrate for Pin1. Hart, S. A., Sabat, M., and Etzkorn, F. A., J. Org. Chem. 63 (1998) 7580; Hart, S. A., and Etzkorn, F. A., J. Org. Chem. 64 (1999) 2298. Synthesis of the Boc-Ser-Ψ[(Z)CH═C]-Pro mimic proceeded with regio- and enantio-selectivity through a [2,3]-sigmatropic rearrangement. Wang, X. J., Hart, S. A., Xu, B., Mason, M. D., Goodell, J. R., and Etzkorn, F. A. (2003), Serine-cis-proline and Serine-trans-proline Isosteres: Stereoselective Synthesis of (Z)- and (E)-Alkene Mimics by Still-Wittig and Ireland-Claisen Rearrangements, J. Org. Chem. 68, 2343-2349.

The possibility of Pin1 activity led to interest and work on certain alkene mimics. (Wang, supra); Wang, X. J., Xu, B., Mullins, A. B., Neiler, F. K., and Etzkorn, F. A. (2004), Conformationally Locked Isostere of PhosphoSer-cis-Pro Inhibits Pin1 23-Fold Better than PhosphoSer-trans-Pro Isostere, J. Am. Chem. Soc. 126, 15533-15542.

However, relatively few inhibitors of Pin1 are known, and Pin1 inhibitors with greater inhibitory activity would be desirable for medical applications. (Hennig, supra); Uchida, T., Takamiya, M., Takahashi, M., Miyashita, H., Ikeda, H., Terada, T., Matsuo, Y., Shirouzu, M., Yokoyama, S., Fujimori, F., and Hunter, T. (2003), Pin1 and Par14 peptidyl prolyl isomerase inhibitors block cell proliferation, Chem. Biol. 10, 15-24.

The reversible phosphorylation of proteins is the most important posttranslational modification that occurs in the cell. It is also the most efficient and versatile signal of intermolecular communication. As a result, many drug targets show high-affinity interactions with phosphorylated molecules, while their unphosphorylated counterparts are not stable for binding to the targets. However, there is a problem for these phosphorylated molecules: unprotected phosphorylated compounds are not effective at penetrating cell membranes, thus are not bioactive because of the negative charges on phosphate groups. One general approach to this problem involves masking the phosphate in a form that neutralizes their negative charges. Among the reversibly masking phosphate compounds, a bis-pivaloyloxymethyl strategy is especially useful since such compounds are quite stable in buffer and plasma and they are readily transformed to free phosphate inside various cell types. Scheme 1 below shows the mechanism for degradation of bis(POM) phosphate inside cells.

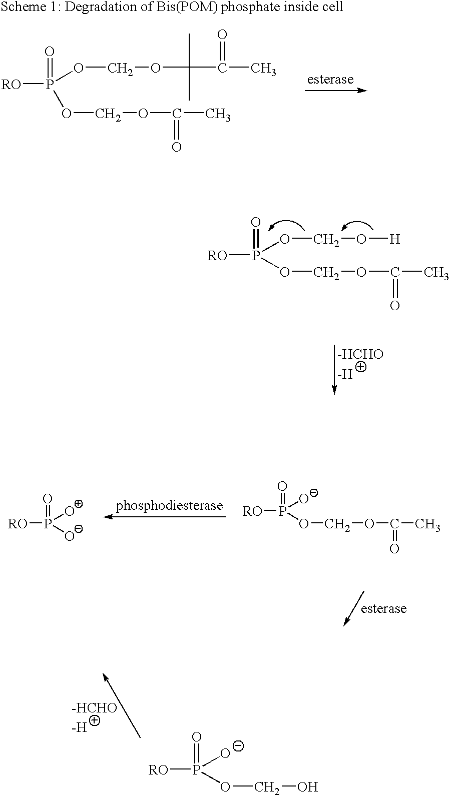

Scheme 1: Degradation of Bis(POM) phosphate inside cell During the process, two different degradation enzymes are involved: esterase and phosphodiesterase. Thus, after the cell entry, the mask for the phosphate group is removed and the compounds converted to a biologically active form. Three methods have been described to introduce the bispivaloyloxymethyl(POM) phosphate triesters. Scheme 2 below shows three methods.

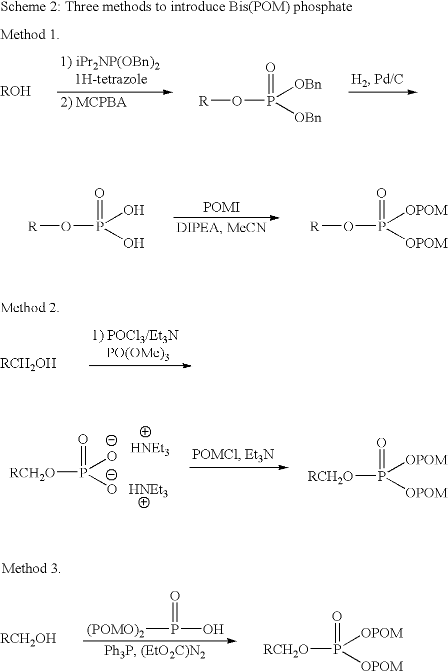

Scheme 2: Three methods to introduce Bis(POM) phosphate

### SUMMARY OF THE INVENTION

The invention in one preferred embodiment provides an alkene compound, selected from the group consisting of: Ac-Phe-Tyr-phosphoSer-Ψ[CH═C]-Pro-Arg-NH₂; Fmoc-bis(pivaloylmethoxy)phosphoSer-Ψ[CH═C]-Pro-2-aminoethyl-(3-indole); Phospho-(D)-serine mimic Ac-Phe-Tyr-phospho-(D)-Ser-Ψ[CH═C]-Pro-Arg-NH₂ and Phospho-(D)-serine mimic Fmoc-bis(pivaloylmethoxy)phospho-(D)-Ser-Ψ[CH═C]-Pro-2-aminoethyl-(3-indole); wherein Ψ means a pseudo amide. In inventive phospho compounds, (Z) stereochemistry is preferred, such as, e.g., Ac-Phe-Tyr-phosphoSer-Ψ[(Z)CH═C]-Pro-Arg-NH₂ and Fmoc-bis(pivaloylmethoxy)phosphoSer-Ψ[(Z)CH═C]-Pro-2-aminoethyl-(3-indole). Other preferred examples of inventive phospho compounds, are, e.g., a compound having inhibitory activity against Pin1; a compound inhibiting the PPIase activity of Pin1; a compound that inhibits the growth of cancer cells; etc.

In another preferred embodiment, the invention provides an alkene compound, wherein the alkene compound is selected from the group consisting of:

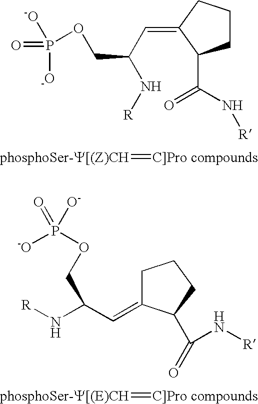

wherein R is a carbonyl group attached to the amine as an amide, and R′ is an amine attached to the carbonyl as an amide.

Another preferred embodiment of the invention provides an alkene compound which is a phospho-(D)-serine mimic, wherein the phospho-(D)-serine mimic is selected from the group consisting of:

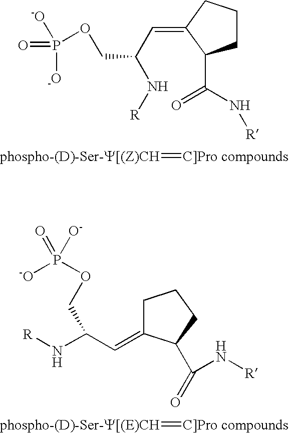

wherein R is a carbonyl group attached to the amine as an amide, and R′ is an amine attached to the carbonyl as an amide.

In the inventive alkene compounds, optionally the phosphate group is masked as a bis(POM) phosphotriester, such as, e.g., the following alkene compounds:

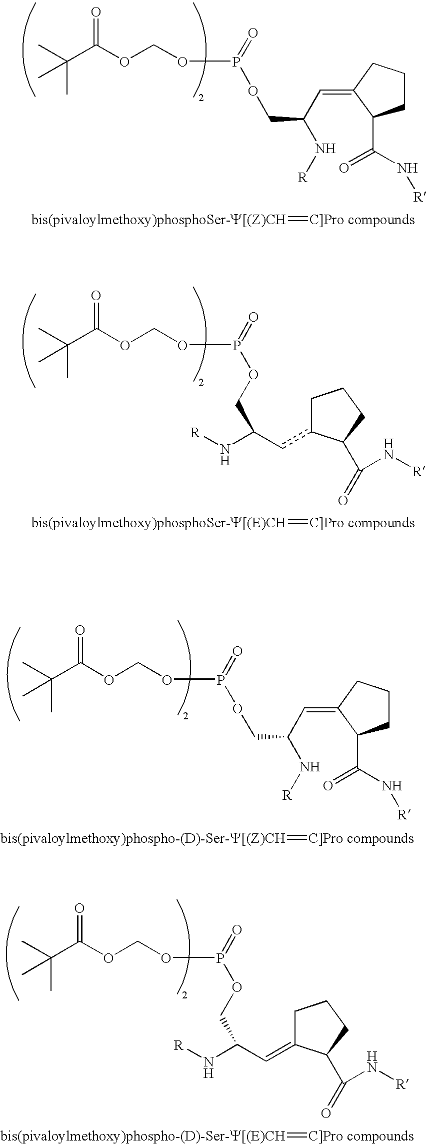

Another preferred embodiment of the invention provides a phosphate mimic modified compound comprising an alkene compound (such as, e.g., any of the above-mentioned alkene compounds) modified with at least one phosphate mimic (such as, e.g., phosphonate, difluorophosphonate, and bis(pivaloylmethoxy) mimics), such as, e.g., the following phosphate mimic modified compounds:

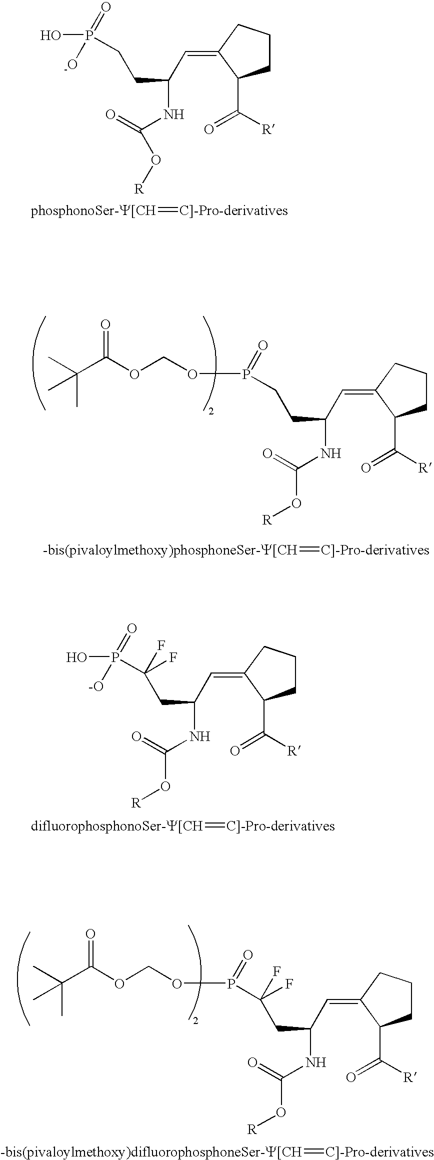

wherein R is a carbonyl group attached to the amine as an amide, and R′ is an amine attached to the carbonyl as an amide.

In all the above formulae where R has been mentioned, preferred examples of R are, e.g., the following 26 acid and acid chloride synthons:

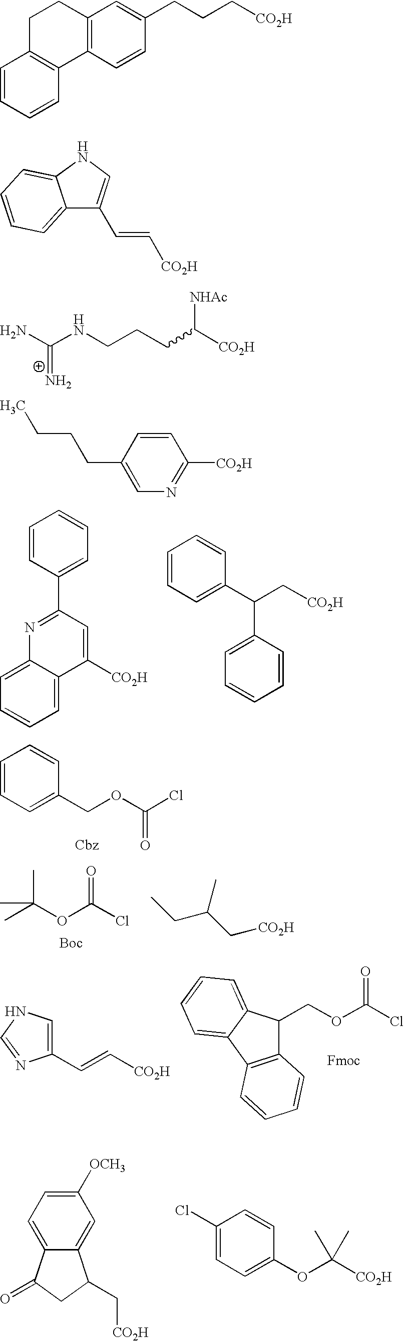

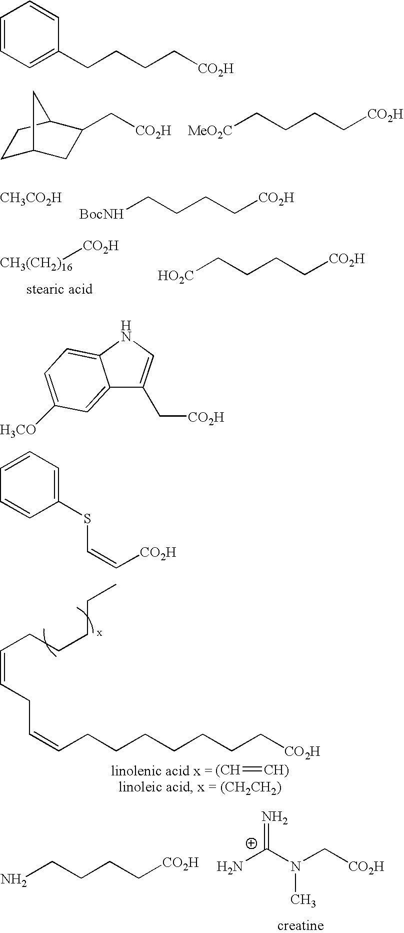

In all the above formulae where R′ has been mentioned, preferred examples of R′ are, e.g., the following 30 amine synthons:

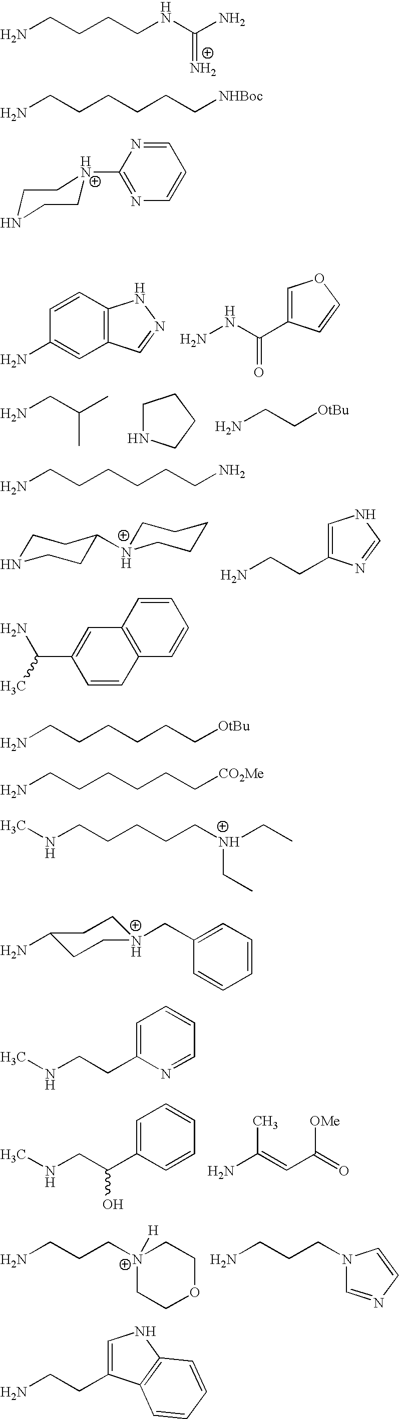

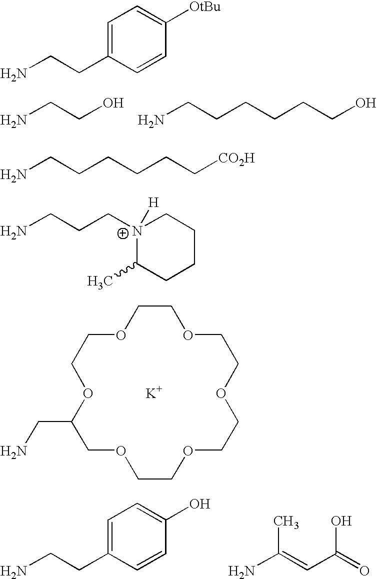

### DETAILED DESCRIPTION OF A PREFERRED EMBODIMENT OF THE INVENTION

The inventive compounds Ac-Phe-Tyr-phosphoSer-Ψ[(Z)CH═C]-Pro-Arg-NH₂ and Fmoc-bis(POM)phosphoSer-Ψ[(Z)CH═C]-Pro-2-aminoethyl-(3-indole) (wherein “Ψ” means a pseudo amide and “POM” means pivaloylmethoxy) are represented by the following formulae (I) and (II) respectively:

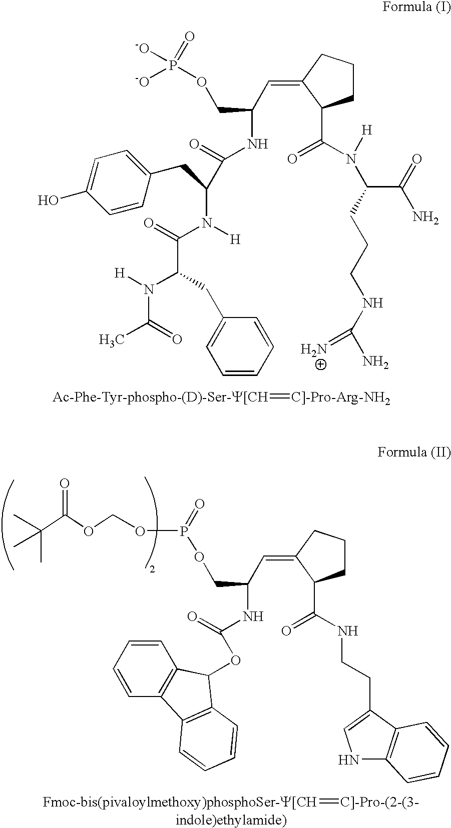

The invention also provides phospho-(D)-serine analogues of Ac-Phe-Tyr-phosphoSer-Ψ[(Z)CH═C]-Pro-Arg-NH₂ and Fmoc-bis(POM) phosphoSer-Ψ[(Z)CH═C]-Pro-2-aminoethyl-(3-indole). Examples of inventive phospho-(D)-serine mimics are, e.g.,

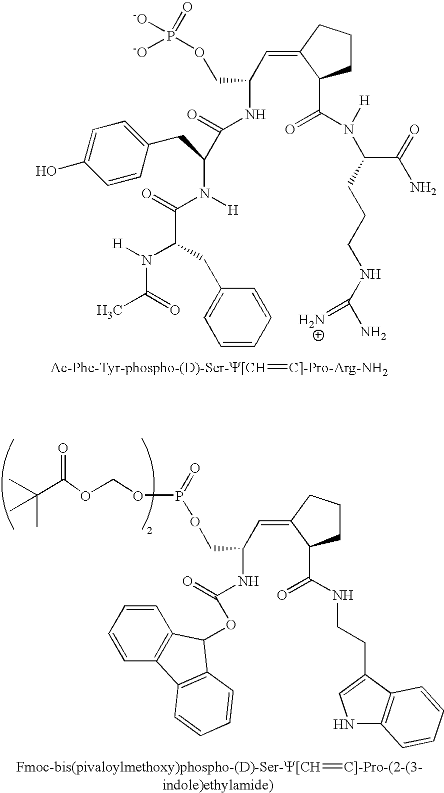

Ac-Phe-Tyr-phosphoSer-Ψ[CH═C]-Pro-Arg-NH₂ and Fmoc-bis(POM)phosphoSer-Ψ[CH═C]-Pro-2-aminoethyl-(3-indole), and phospho-(D)-serine analogues of Ac-Phe-Tyr-phosphoSer-Ψ[CH═C]-Pro-Arg-NH₂ and Fmoc-bis(POM)phosphoSer-Ψ[CH═C]-Pro-2-aminoethyl-(3-indole) are useful for their Pin1 activity, and promising for their use against a variety of cancer types, and against addiction, especially cocaine addiction.

Pin1 is different from other cell cycle regulators that belong mainly to the abundant classes of kinases, phosphatases, histone acetyl transferases and histone deactetylases. Because of the unique chemical mechanism of Pin1, a high degree of specificity may be obtained with specific inhibitors. Thus, new and better specific inhibitors of Pin1 that are peptidomimetic are advantageous. Thus, inventive compounds (such as phospho compounds, alkene compounds, etc. of this invention) having inhibitory activity against Pin1 (such as compounds that inhibit the PPIase activity of Pin1) are preferred. Inhibitory activity against Pin1 having been observed experimentally (see, e.g., experimental data herein), the present invention further provides a method of inhibiting activity against Pin1, comprising administration of an effective amount of any of the inventive compounds herein to a subject, wherein Pin1 activity is inhibited.

Inventive compounds (such as phospho compounds, alkene compounds, etc.) that inhibit the growth of cancer cells also are particularly preferred. The present invention further provides a method of inhibiting the growth of cancer cells, comprising administration of an effective amount of any of the inventive compounds herein to a subject having cancer cells, wherein growth of the cancer cells is inhibited. Administration could take place by a number of different routes including oral, intravenous, intrapertoneal, intramuscular, subcutaneas, sublingual, aerosol delivery, etc. The compounds of the present invention may be formulated with a variety of carriers (e.g., oils or aqueous based), stabilizers, emulsifiers, preservatives, as well as other compounds having pharmaceutical activity, as may be desirable for the particular application.

The following examples are for better appreciating the invention, and the invention is not limited thereto.

### Example 1

### Ac-Phe-Tyr-phosphoSer-Ψ[(Z)CH═C]-Pro-Arg-NH₂

The IC50 value for the inhibition of human Pin1 peptidyl-prolyl isomerase activity was measured to be 0.97+/−0.09 μM. The Ser unprotected substrate analogue was synthesized according to the following reaction Scheme 3:

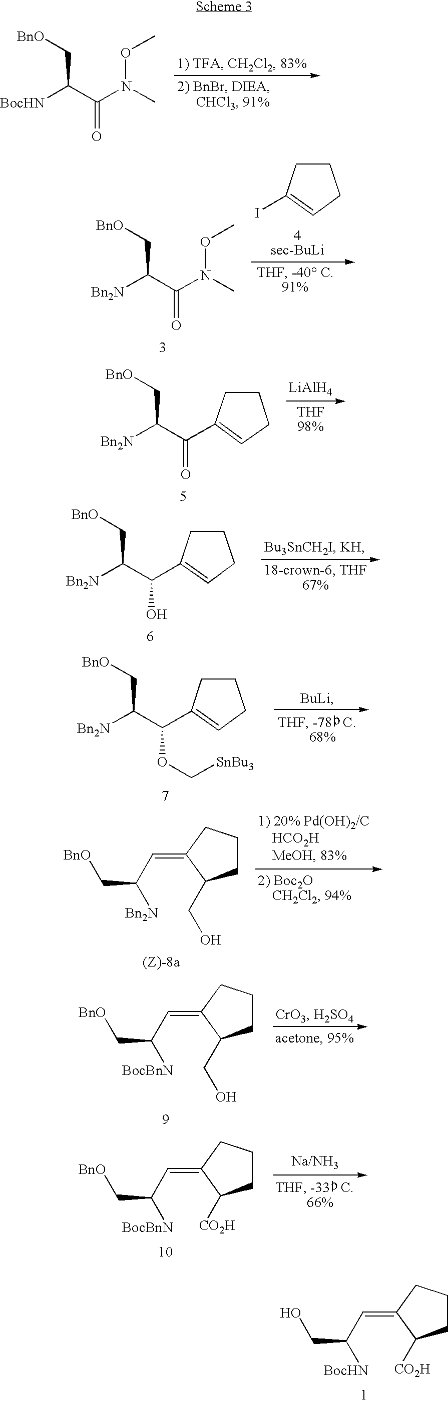

Two new amide isosteres of Ser-cis- and -trans-Pro dipeptides were designed and stereoselectively synthesized. These amide isosteres were incorporated into inhibitors of the phosphorylation-dependent Pin1. The cis mimic, the (Z)-alkene isomer, was formed by a Still-Wittig [2,3]-sigmatropic rearrangement. The trans mimic, the (E)-alkene, was synthesized by an Ireland-Claisen [2,3]-sigmatropic rearrangement. Starting from Boc-Ser(OBn)-OH, both mimics were synthesized in Boc-protected form suitable for peptide synthesis with an overall yield of 20% in 10 steps for the cis mimic and 13% in eight steps for the trans mimic.

Peptidomimetics of cis- and -trans-prolines were reviewed. One of the ideal peptide bond surrogates is the alkene because of the similar geometrical disposition of the substituents attached to either of these functional groups. Fluoroalkene isosteres of Ala-trans-Pro were reported previously. An (E)-alkene trans-Pro mimic was synthesized by others previously and shown to inhibit the PPIase activity of FKBP, but that mimic included an extra methyl group not wanted by the present inventors. Relatively fewer Z-alkenes had been made due to the difficulty of (Z)-alkene formation and the possibility of isomerization of the β,γ-unsaturated carbonyl to the more stable α,β-unsaturated carbonyl compounds. In this Example, a Ser-cis-Pro (Z)-alkene was synthesized. The alkene isostere was an amide bond surrogate and the alkene was not a substrate for the peptidyl-prolyl isomerase.

Optically active amino acids are versatile synthons for stereoselective synthesis. Starting with the optically pure amino acid N-terminal to Pro provides the source for stereoselective synthesis, and also imparts generality to the synthesis of any Xaa-Pro alkene mimic. In this Example, N-Boc-O-benzyl-L-serine, used in Merrifield peptide synthesis, was chosen as the starting material for the syntheses of both Ser-cis-Pro and Ser-trans-Pro mimics. Because Ser is so highly functionalized, significant challenges and side reactions were encountered during the synthesis of these particular Pro mimics. A Still-Wittig [2,3]-sigmatropic rearrangement could be used to form both the (Z)- and (E)-alkene stereoisomers, but the (E)-alkene was synthesized best by an Ireland-Claisen [3,3]-sigmatropic rearrangement.

Still-Wittig Route to Ser-cis-Pro Mimic. The key steps in the synthesis of Boc-Ser-Ψ[(Z)CH═C]-Pro-OH were stereoselective reduction of the ketone to the (S,S)-alcohol, and Still-Wittig rearrangement to the (Z)-alkene. Starting with the Weinreb amide of Boc-Ser(OBn)-OH, the ketone was formed by condensation with cyclopentenyl lithium derived from cyclopentenyl iodide. The 1-iodocyclopentene reagent was prepared in two steps with 50% overall yield from cyclopentanone most cleanly by the method of Barton. Reduction of the ketone with LiAlH₄ proceeded with Felkin-Ahn stereoselectivity to give an (S,S)-alcohol. A single diastereomer was observed in the NMR spectra. The absolute stereochemistry was demonstrated by derivatization of a mixture of diastereomers as the oxazolidonones and measurement of the ¹H NMR coupling constants. The iodomethyltributyl tin reagent was prepared by the method of Steitz et al. Fractional distillation is recommended. After forming the intermediate tributylstannylmethyl ether, Still-Wittig rearrangement gave a 68% Z to 25% E ratio of alkene. The diastereomers were readily separated by column chromatography. The geometry of the alkenes was determined by ID-NOE.

Taking the (Z)-alkene, the benzyl protection was removed and the amine was reprotected for peptide synthesis. (Alternative amine protection, such as trityl or Boc, gave poor stereoselectivity and/or yields in some previous reactions.) The first benzyl of the amine was selectively removed by hydrogenation with formic acid on Pearlman's catalyst in the presence of the benzyl ether and the alkene. Boc protection was required for removing the second benzyl. At this stage, it was possible to remove the second benzyl by sodium/ammonia reduction, but Jones oxidation of the resulting alcohol, with only Boc still protecting the amine, gave extremely poor yields. Jones oxidation on the doubly-protected Boc-benzyl amine produced an acid in 95% yield. Initially, a major side product from the Jones oxidation was a ketone, probably resulting from allylic oxidation and C—C bond cleavage. This side product was minimized by adding an excess of the Jones reagent to the alcohol and keeping the reaction at 0° C. Final benzyl deprotection by Na/NH₃, reduction yielded Boc-Ser-Ψ[(Z)CH═C]Pro-OH. A large excess of sodium was required to prevent the cyclization of the side chain oxy-anion onto the Boc carbonyl to produce a cyclic carbamate. Presumably benzyl ether deprotection is slightly more rapid than benzyl amine deprotection and the large excess of sodium increases the rate for both to improve the yield of the desired product of this Example.

Still-Wittig Route to (E)-Alkene Ser-trans-Pro Mimic. The (E)-form of this Example bears the opposite stereochemistry (S) at the allylic position of the cyclopentyl ring necessary to mimic L-Pro. In the context of amino acid stereochemistry, the (R,E,S)-form in this Example is the precursor to an L-Ser-trans-D-Pro mimic, while the (R,Z,R)-form leads to the L-Ser-cis-L-Pro mimic. Compounds including the L-Ser-trans-D-Pro form of the mimic may also find use in Pin1 inhibition and anti-cancer activity.

### Example 2

In order to mimic the structure of naturally occurring amino acids, the (R,E,R) mimic of L-Ser-trans-L-Pro was used in this Example.

Ireland-Claisen Route to (E)-Alkene Ser-trans-Pro Mimic. The Ireland-Claisen rearrangement was more successful than the Still-Wittig rearrangement at producing the (E)-alkene of this Example, both in stereoselectivity and in yield. The Weinreb amide was prepared easily from N-Boc-O-benzyl-L-serine. The reaction of the Weinreb amide with cyclopentenyl lithium gave the desired ketone in 86% yield (by adding three equivalents of cyclopentenyl lithium in portions). The chelation-controlled Luche reduction of the ketone gave a pair of diastereomers in good yield (92%) and stereoselectivity (4:1). The major diastereomer was the (S,R) form by derivatization as the oxazolidinones.

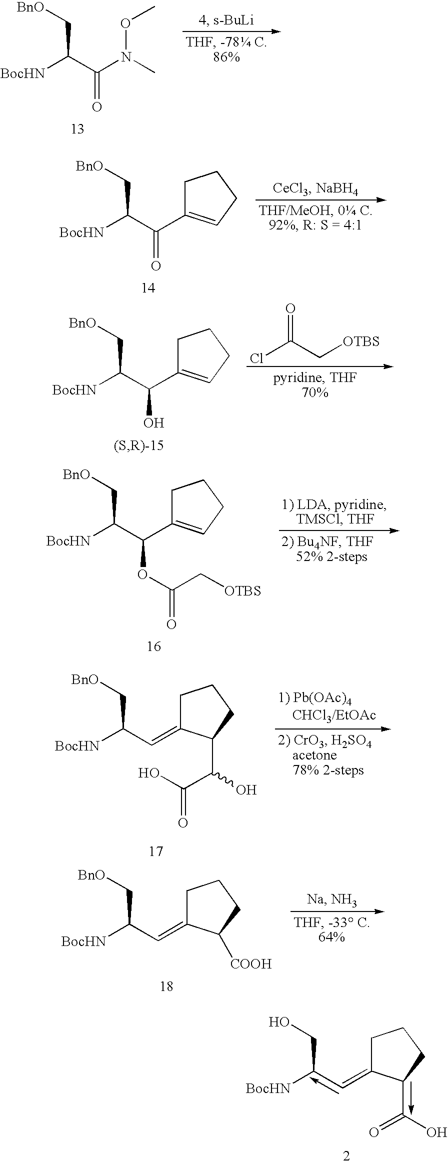

The alcohol was transformed readily to the Ireland-Claisen precursor ester by reaction with t-butyldimethylsilyloxyacetyl chloride. The Ireland-Claisen rearrangement of the ester was the key step in the synthesis of the Ser-trans-Pro mimic. Activation of TMSCl by pyridine was necessary. The intermediate TBS-protected alcohol was unstable towards silica gel, but subsequent removal of the TBS protecting group by t-butyl ammonium fluoride (TBAF) in THF in THF gave the α-hydroxy acid as a stable product. The crude ¹H NMR showed three minor diastereomers in addition to the major, desired isomer, but the stereochemistry at the alcohol center is eliminated by oxidation in the next step. The NOESY showed the (E)-alkene as the major product of the rearrangement.

In the oxidation to cleave one carbon and oxidize the resulting product in this Example, lead (IV) tetraacetate was used to give clean and quantitative β,γ-unsaturated aldehyde product.

The β,γ-unsaturated aldehyde of this Example was carried on to oxidation without further purification. Isomerization of the β,γ-unsaturated aldehyde to the more stable α,β-unsaturated aldehyde occurred readily during basic work up (aqueous NaHCO₃) or silica gel purification. Jones oxidation of the aldehyde yielded the corresponding β,γ-unsaturated carboxylic acid, without loss of the acid sensitive Boc group. The side product from allylic oxidation was not observed in this oxidation of the aldehyde. The β,γ-unsaturated acid in this Example is stable towards isomerization under aqueous acidic or basic conditions. The (E)-alkene stereochemistry of the β,γ-unsaturated acid of this Example was demonstrated by NOESY. The benzyl protection on oxygen was successfully removed with Na/NH₃ to give Boc-Ser-Ψ-[(E)CH═C]Pro-OH.

### Example 3

The specificity of Pin1 is fairly broad outside of the phosphoSer-Pro dipeptide (Table 1). Based on the affinity of Pin1 for cdc25 wild type and Thr mutants, the probable sites of Pin1 isomerization of cdc25 as the substrate are listed in Table 2 for both Xenopus and by analogy, human cdc25.

TABLE 1
Substrate specificity of Pin1, antibody ligands for MPM-2
and sequence of probable Pin1 substrate sites in cdc25.

| | Ligand Position | | | | | | |
|---|---|---|---|---|---|---|---|
| | −4 | −3 | −2 | −1 | +1 | +2 | +3 |
| Pin1(a): | W | F | Y | pS | P | R | L |
| | Y | I | R | | | F | I |
| | F | | F | | Y | | |
| | W | | W | | | | |
| MPM-2: | Y | W | F | pS | P | L | X |
| | F | F | L | | | Y | |
| | W | | I | | | V | |

TABLE 2
Sequence of probable Pin1 substrate sites in Xenopus and human
cdc25.

| | Ligand Position | | | | | | |
|---|---|---|---|---|---|---|---|
| | −4 | −3 | −2 | −1 | +1 | +2 | +3 |
| Xenopus cdc25(a): | Q | P | L | pT | P | V | T |
| Xenopus cdc25(a): | S | G | E | pT | P | K | R |
| Human cdc25: | V | P | R | pT | P | V | G |

Part of the first peptide sequence listed in Table 1 was synthesized with the phosphoSer-Pro alkene mimics. Additional substrate analogs are made with a variety of amino acids, both natural and unnatural, as well as other functional groups. In addition to the alkene mimics, a variety of phosphate analogs are synthesized, including phosphate, phosphonate, difluorophosphonate, and their bis(pivaloylmethoxy) mimics. Examples of phosphate mimics of Ser-cis and trans-Pro are, e.g.,

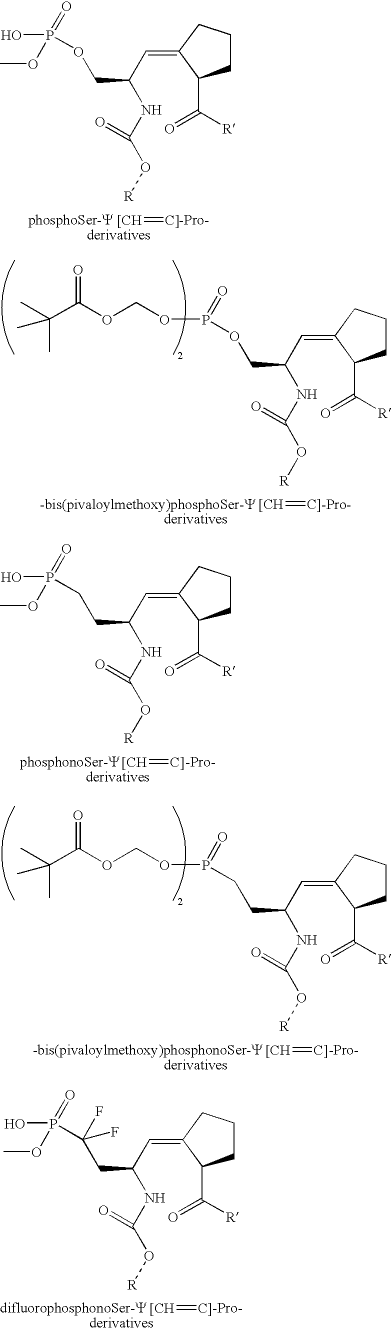

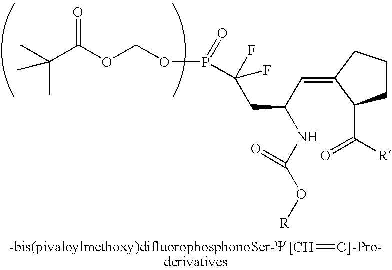

### Example 4

### Experiment

General. Unless otherwise indicated, all reactions were carried out under N₂ in flame-dried glassware. THF, toluene, and CH₂Cl₂ were dried by passage through alumina. Anhydrous (99.8%) DMF was purchased from Aldrich and used directly from SureSeal™ bottles. Dimethyl sulfoxide (DMSO) was anhydrous and dried with 4 Å molecular sieves. Triethylamine (TEA) was distilled from CaH₂ and (COCl)₂ was distilled before use each time. Diisopropylethylamine (DIEA) was distilled from CaH₂ under a N₂ atmosphere. Brine (NaCl), NaHCO₃ and NH₄Cl refer to saturated aqueous solutions unless otherwise noted. Flash chromatography was performed on 32-63 μm or 230-400 mesh, ASTM silica gel with reagent grade solvents. Melting points were uncorrected. NMR spectra were obtained at ambient temperature in CDCl₃ unless otherwise noted. Proton (300 MHz) NMR spectra were obtained for compounds 1, 3, 6, and 8-12, and carbon-13 (75 MHz) for compounds 1-6 and 8-12. Proton (500 MHz) NMR spectra were obtained for compounds 2, 4, 5, 7-9 and 13-18, and carbon-13 (125 MHz) for compounds 7 and 13-18.

N,N,O-Tribenzyl Serine Weinreb amide (3). N-Boc-O-benzyl serine Weinreb amide (24.1 g, 71.2 mmol) was dissolved in CH₂Cl₂ (400 mL) and TFA (125 mL) was added and stirred 30 min. The mixture was concentrated, then quenched with NaHCO₃ until gas evolution ceased. The aqueous mixture was extracted with CH₂Cl₂ (8×300 mL), dried on MgSO₄, and concentrated. Chromatography on silica with 50% EtOAc in petroleum ether (pet. ether) to remove impurities, followed by product elution with 10% MeOH in EtOAc yielded 13.1 g (83%) of the amine as a clear oil. ¹H NMR δ 7.40-7.20 (m, 5H), 4.57 (d, J=12.1, 1H), 4.52 (d, J=12.1, 1H), 4.06 (m, 1H), 3.67 (s, 3H), 3.66-3.45 (m, 2H), 3.20 (s, 3H), 1.88 (br s, 2H). The amine (13 g, 58 mmol) was dissolved in CH₂Cl₂ (50 mL), then benzyl bromide (24.8 g, 145 mmol) and DIEA (37.4 g, 290 mmol) were added. After 4 d at rt, the reaction was diluted with EtOAc (600 mL), washed with NH₄Cl (4×200 mL) and brine (200 mL), dried on MgSO₄, and concentrated. Chromatography on silica with 10% EtOAc in pet. ether to remove benzyl bromide, then 50% EtOAc in pet. ether to elute the product yielded 21.4 g (91%) of dibenzyl amine 3 as a clear oil. ¹H NMR δ 7.40-7.17 (m, 15H), 4.56 (d, J=11.9, 1H), 4.48 (d, J=11.9, 1H), 4.13 (m, 1H), 3.98-3.84 (m, 4H), 3.76 (d, J=14.1, 2H), 3.28 (br s, 3H), 3.20 (br s, 3H). ¹³C NMR δ 171.5, 140.0, 138.2, 128.7, 128.1, 127.9, 127.3, 126.6, 73.0, 68.6, 60.8, 56.4, 55.0, 30.9. Anal. calcd for: C₂₆H₃₀N₂O₃: C, 74.61; H, 7.22; N, 6.69, found: C, 74.31; H, 7.32; N, 6.40.

1-Iodocyclopentene (4). (By the method of Barton et al.²⁵) Cyclopentanone (44 ml, 0.50 mol) and hydrazine monohydrate (115 mL, 2.37 mol) were combined at rt and heated at reflux for 16 h. The reaction was poured into water (500 mL) and extracted with CH₂Cl₂ (4×200 mL), washed with brine (200 mL), dried over Na₂SO₄ and concentrated to give 40 g (80%) of the hydrazone as a colorless liquid. ¹H NMR δ 4.80 (s, 2H), 2.33-2.30 (t, 2H), 2.16-2.12 (m, 2H), 1.85-1.67 (m, 4H). To a solution of 12 (97.5 g, 384 mmol) in Et₂O (600 mL) was added a solution of tetramethylguanidine (265 mL, 2.09 mol) in Et₂O (400 mL) slowly (Caution: exothermic!) and stirred for 2.5 h. A solution of cyclopentanone hydrazone (17.3 g, 174 mmol) in Et₂O (200 mL) was added dropwise over 2.5 h (Caution: exothermic!) and stirred for 16 h, then heated at reflux for 2 h. The reaction was cooled to rt filtered to remove the solids and concentrated to remove Et₂O. The solution was reheated at 80-90° C. for 3 hr. The reaction was cooled to rt, diluted with Et₂O (500 mL), washed with 2 N HCl (3×150 mL), Na₂S₂O₃ (3×100 mL), NaHCO₃ (100 mL), brine (100 mL), dried over MgSO₄ and concentrated to give 21.1 g (62%) of 4 as a pale yellow liquid that was stored under N₂ at −20° C. and used without further purification, usually within a week of synthesis. (The product may be purified, if necessary, by chromatography with petroleum ether on silica.) ¹H NMR δ 6.12-6.10 (m, 1H), 2.64-2.58 (m, 2H), 2.36-2.30 (m, 2H), 1.98-1.90 (m, 2H).

Ketone (5). Cyclopentenyl lithium was generated by adding fresh s-BuLi (1.3 M in cyclohexane, 50 mL, 65 mmol) to a solution of freshly prepared 1-iodocyclopentene 4 (10.0 g, 51.5 mmol) in THF (100 mL) at −40° C. The solution was maintained at −40° C. for 70 min, and Weinreb amide 3 (7.40 g, 17.7 mmol) in THF (30 mL) was cooled to −40° C. and added slowly via cannula. The mixture was stirred 1 h at −40° C. The reaction was quenched with NH₄Cl (20 mL), diluted with EtOAc (600 mL), washed with NH₄Cl (3×100 mL), brine (100 mL), dried over Na₂SO₄, and concentrated. Chromatography on silica with 5% EtOAc in hexanes yielded 7.1 g (94%) of the ketone 5. ¹H NMR δ 7.39-7.20 (m, 15H), 6.11 (m, 1H), 4.55 (d, J=12.3, 1H), 4.48 (d, J=12.3, 1H), 4.24 (app. t, J=6.6, 1H), 3.90 (d, J=6.6, 2H), 3.79 (d, J=13.6, 2H), 3.71 (d, J=14.1, 2H), 2.59-2.39 (m, 4H), 1.98-1.84 (m, 2H). ¹³C NMR δ 197.8, 145.5, 144.7, 139.7, 138.2, 128.8, 128.2, 128.1, 127.5, 126.9, 73.3, 67.6, 60.6, 54.8, 33.9, 30.5, 22.6. Anal. calcd for: C₂₉H₃₁NO₂: C, 81.85; H, 7.34; N, 3.29, found: C, 81.51; H, 7.42; N, 3.52.

(S,S)-Alcohol (6). Ketone 5 (6.8 g, 16 mmol) was dissolved in THF (250 mL) and LiAlH₄ (6.0 g, 160 mmol) was added. After 1 h, the reaction was quenched with MeOH (50 mL), then NH₄Cl (50 mL), diluted with EtOAc (500 mL), washed with NH₄Cl (150 mL), and 1 M sodium potassium tartrate (2×150 mL). The aqueous layers were extracted with CH₂Cl₂ (3×200 mL). The combined organic layers were dried over MgSO₄ and concentrated to yield 6.68 g (98%) of alcohol 6 as a colorless oil. ¹H NMR δ 7.49-7.24 (m, 15H), 5.65 (m, 1H), 4.62 (d, J=11.9, 1H), 4.53 (d, J=11.9, 1H), 4.48 (s, 1H), 4.26 (d, J=10.1, 1H), 4.02 (d, J=13.2, 2H), 3.80-3.70 (m, 3H), 3.58 (dd, J=10.6, 3.1, 1H), 3.07 (m, 1H), 2.43-2.17 (m, 3H), 2.00-1.75 (m, 3H). ¹³C NMR δ 144.1, 139.0, 138.2, 129.2, 129.0, 128.3, 127.5, 127.4, 127.1, 73.2, 67.5, 66.4, 59.7, 54.3, 32.0, 29.5, 23.0. Anal. calcd for: C₂₉H₃₃NO₂: C, 81.46; H, 7.78; N, 3.28, found: C, 81.25; H, 7.66; N, 3.11.

Stannane (7). To a solution of alcohol 6 (2.20 g, 5.15 mmol) in THF (40 mL), was added 18-crown-6 (4.09 g, 15.5 mmol) in THF (10 mL), KH (1.03 g, 7.73 mmol, 35% suspension in mineral oil) in THF (10 mL), and Bu₃SnCH₂I,²⁹ purified by fractional distillation at reduced pressure, (3.33 g, 7.73 mmol) in THF (10 mL), and stirred 30 min at rt. The reaction was quenched with MeOH and diluted with EtOAc (400 mL), washed with NH₄Cl (2×100 mL), brine (100 mL), dried on MgSO₄, and concentrated. Purification by chromatography on silica with 3% EtOAc in hexanes yielded 3.51 g (94%) of stannane 7 as a clear liquid. ¹H NMR δ 7.40-7.26 (m, 15H), 5.60 (br s, 1H), 4.45 (d, J=12.0, 1H), 4.37 (d, J=12.0, 1H), 4.05 (d, J=7.8, 1H), 3.99 (d, J=13.7, 2H), 3.83 (d, J=13.7, 2H), 3.74 (dm, J=9.9, 1H), 3.60 (dd, J=9.6, 5.7, 1H), 3.53 (dd, J=9.6, 4.6, 1H), 3.41 (dm, J=9.6, 1H), 2.99 (m, 1H), 2.40-2.28 (m, 2H), 1.99 (br s, 2H), 1.82 (m, 2H), 1.54 (m, 6H), 1.33 (m, 6H), 0.91 (m, 15H). ¹³C NMR (125 MHz) δ 143.0, 141.6, 138.9, 129.1, 128.6, 128.4, 128.0, 127.6, 126.5, 85.4 (s), 85.4 (d, J_{C-}=51), 73.2, 70.6, 59.2, 58.6, 55.8, 32.3, 31.1, 29.4 (s), 29.4 (d, J_{C—Sn}=20), 27.6 (s), 27.6 (d, J_{C—Sn}=54), 23.5, 13.9, 9.0 (s), 9.0 (dd, J_{CSn}=316, 7.6).

(Z)-Alkene 8a and (E)-alkene 8b. Stannane 7 (9.60 g, 13.1 mmol) was dissolved in THF (150 mL) and cooled to −78° C. n-BuLi (2.5 M in hexane, 15 mL, 39 mmol) was cooled to −78° C., added slowly via cannula and stirred 1.5 h at −78° C. The reaction was quenched with MeOH and concentrated. The residue was diluted with EtOAc (700 mL), washed with NH₄Cl (2×150 mL, brine (150 mL), dried on Na₂SO₄ and concentrated. Chromatography on silica with 15% EtOAc in hexanes yielded 3.0 g (53%) of (Z)-8a, and 1.57 g (28%) of (E)-8b as clear oils. (NOE spectra are included in Supporting Information of the preliminary communication.¹⁹) (E)-8b: ¹H NMR δ 7.38-7.27 (m, 15H), 5.43 (br d, J=9.4, 1H), 4.51 (d, J=12.1, 1H), 4.47 (d, J=12.1, 1H), 3.84 (d, J=13.9, 2H), 3.73 (m, 1H), 3.64-3.47 (m, 6H), 2.65 (m, 1H), 2.05 (m, 2H), 1.85 (m, 1H), 1.69 (m, 1H), 1.56 (m, 2H). ¹³C NMR δ 148.6, 140.3, 138.5, 129.4, 128.5, 128.2, 128.0, 127.5, 127.3, 126.6, 117.6, 72.6, 71.7, 65.4, 57.3, 54.7, 47.0, 29.6, 29.2, 24.1. (Z)-8a: ¹H NMR δ 7.38-7.26 (m, 15H), 5.55 (br d, J=8.7, 1H), 4.57 (d, J=12.2, 1H), 4.53 (d, J=12.2, 1H), 4.12 (br s, 1H), 3.89 (d, J=13.3, 2H), 3.79 (m, 1H), 3.67 (m, 4H), 3.33 (m, 1H), 3.27 (m, 1H), 2.53 (m, 1H), 2.31-2.18 (m, 2H), 1.71-1.47 (m, 4H). ¹³C NMR δ 149.0, 139.2, 138.4, 129.4, 128.3, 128.0, 127.5, 126.8, 120.9, 73.2, 69.7, 64.8, 57.3, 55.0, 43.5, 33.1, 29.4, 23.1. Anal. calcd for: C₃₀H₃₅NO₂: C, 81.59; H, 7.99; N, 3.17, found: C, 81.42; H, 8.27; N, 3.25.

Bocbenzylamine (9). (Z)-Alkene 8 (1.44 g, 3.26 mmol), and 20% Pd(OH)₂/C (150 mg) were blanketed with Ar and MeOH (100 ml) was added, followed by 96% HCOOH (20 ml). After stirring exactly 20 min, the reaction was filtered immediately through Celite, concentrated, neutralized with solid NaHCO₃ until gas evolution ceased, extracted with CH₂Cl₂ (5×100 ml), dried over Na₂SO₄, and concentrated to yield 1.1 g (98%) of the monobenzylamine without further purification. ¹H NMR (500 MHz) δ 7.36-7.30 (m, 100H), 5.50 (br d, J=8.3, 1H), 4.56 (br d, J=1.6, 2H), 3.72 (d, J=11.2, 1H), 3.66-3.60 (m, 3H), 3.55-3.50 (m, 1H), 3.48-3.45 (dd, J=10.8, 4.3, 1H), 3.41-3.37 (m, 1H), 2.83 (m, 1H), 2.37-2.22 (m, 2H), 1.89-1.85 (m, 1H), 1.64 (m, 1H), 1.54-1.38 (m, 2H). HRMS (FAB⁺) calcd for C₂₃H₃₀NO₂ (M+1)⁺ 352.2276, found 352.2278. The monobenzylamine (1.10 g, 3.12 mmol) was dissolved in CH₂Cl₂ (60 ml), and di-t-butyldicarbonate (1.70 g, 7.79 mmol) was added and stirred for 17 h. The mixture was concentrated and purification by chromatography on silica with 20% EtOAc in hexanes yielded 1.3 g (95%) of the Bocbenzyl amine 9 as a pale yellow oil. ¹H NMR (500 MHz) δ 7.36-7.16 (m, 10H), 5.36 (br d, J=8.9, 1H), 5.18 (br s, 1H), 4.47-4.37 (m, 4H), 3.48-3.46 (m, 5H), 2.87 (br s, 1H), 2.20 (m, 2H), 1.75 (m, 1H), 1.65 (m, 2H), 1.54 (m, 1H), 1.34 (br s, 9H). ¹³C NMR δ 155.7, 149.2, 139.9, 138.1, 127.9, 127.8, 127.2, 126.7, 126.3, 117.8, 79.8, 72.3, 71.1, 64.3, 54.1, 47.3, 43.9, 33.2, 29.1, 28.0, 23.0. HRMS calcd for C₂₈H₃₇NO₄ (MH⁺) m/z=452.2801, found m/z=452.2813.

Bocbenzylamino acid (10). Bocbenzyl amine 9 (2.2 g, 4.9 mmol) was dissolved in acetone (220 mL) and cooled to 0° C. Jones reagent (2.7 M H₂SO₄, 2.7 M CrO₃, 4.5 mL, 12 mmol) was added and stirred 30 min at 0° C. The reaction was quenched with isopropanol (50 mL) and stirred 5 min. The mixture was diluted with water (400 mL), extracted with CH₂Cl₂ (10×50 mL), dried on MgSO₄, and concentrated. Chromatography on silica with 20% EtOAc in pet. ether yielded 2.1 g (95%) of the acid 10 as a pale yellow oil. ¹H NMR δ 7.34-7.16 (m, 10H), 5.53 (br d, J=9.2, 1H), 4.92 (br s, 1H), 4.47-4.27 (m, 4H), 3.69-3.24 (m, 3H), 2.46 (m, 1H), 2.28 (m, 1H), 2.11 (m, 1H), 1.89 (m, 2H), 1.62 (m, 1H), 1.38 (br s, 9H). ¹³C NMR (CDCl₃): δ179.1, 155.6, 145.7, 139.8, 138.3, 128.2, 128.1, 127.4, 127.1, 126.6, 120.9, 80.0, 72.6, 72.0, 55.6, 49.0, 45.9, 33.5, 31.0, 28.3, 23.8. HRMS calcd for C₂H₃₆NO₅ (MH⁺) m/z=466.2593, found m/z=466.2601.

Boc-SerΨ[(Z)CH═C]Pro-OH (1). NH₃ (ca. 160 mL) was distilled into 40 mL THF at −78° C. and allowed to warm to reflux (−33° C.). Na (ca. 2.0 g, 87 mmol) was added until a deep blue solution was sustained. A solution of acid 10 (2.0 g, 4.3 mmol) in THF (10 mL) was added directly to the Na/NH₃ solution slowly via cannula over ca. 5 min. After stirring 45 min at reflux, the reaction was quenched with NH₄Cl (10 mL), then allowed to warm to rt with concentration to ca. 30 mL (Caution! NH₃ evolved). The mixture was diluted with NH₄Cl (50 mL), acidified with 1 N HCl to pH 7 and extracted with CHCl₃ (10×50 mL), dried on MgSO₄, and concentrated to give 810 mg (66%) of the alcohol 1 as a pale yellow oil. Further purification can be achieved by chromatography on silica with 3% MeOH in CHCl₃ if desired. ¹H NMR (DMSO-d₆) δ 6.48 (br d, J=6.2, 1H), 5.20 (d, J=8.4, 1H), 4.08 (m, 1H), 3.36 (m, 1H), 3.28 (dd, J=10.6, 5.7, 1H), 3.13 (dd, J=10.6, 6.6, 1H), 2.20 (m, 2H), 1.81 (m, 2H), 1.67 (m, 1H), 1.47 (m, 1H), 1.31 (s, 9H). ¹³C NMR (DMSO-d₆) δ 175.4, 154.8, 142.7, 122.5, 77.4, 64.0, 51.9, 45.5, 33.5, 31.2, 28.3, 24.1. HRMS calcd for C₁₄H₂₃NO₅ (MH⁺) m/z=286.1654, found m/z=286.1653.

Boc-Ser(OBn) Weinreb amide (13). N-Boc-Ser(OBn)-OH (2.95 g, 10.0 mmol), N,O-dimethylhydroxylamine hydrochloride (1.85 g, 20.0 mmol) and DIEA (5.2 g, 40 mmol) were dissolved in 1:1 CH₂Cl₂/DMF (100 mL) and cooled to 0° C. 1-Hydroxy-1H-benzotriazole (HOBt, 1.84 g, 12.0 mmol), DCC (2.48 g, 12.0 mmol) and DMAP (ca. 30 mg) were added and the reaction was stirred for 24 h. The reaction was filtered to remove dicyclohexylurea and concentrated. The resulting slurry was diluted with 150 mL ethyl acetate and washed with NH₄Cl (2×50 mL), NaHCO₃ (2×50 mL) and brine (50 mL). The organic layer was dried on MgSO₄ and concentrated. Chromatography on silica with 30% EtOAc in hexane gave 3.04 g (90%) of 13 as a colorless syrup. ¹H NMR δ 7.35-7.23 (m, 5H), 5.42 (d, J=8.5, 1H), 4.87 (br, s, 1H), 4.56 (d, J=12.5, 1H), 4.49 (d, J=12.5, 1H), 3.71 (s, 3H), 3.66 (m, 2H), 3.17 (s, 3H), 1.43 (s, 9H).

Ketone (14). To a solution of 1-iodocyclopentene 4 (7.59 g, 39.1 mmol) in 100 mL THF at −40° C. was added s-BuLi (1.3 M in cyclohexane, 60 ml, 78 mmol). The reaction was stirred at −40° C. for 3 h to generate cyclopentenyl lithium. Then the mixture was added via syringe in three portions to a solution of Weinreb amide 13 (4.41 g, 13.0 mmol) in THF (50 mL), dried over 3 Å molecular sieves for 3 h, at −78° C. The mixture was stirred for 3 h at −78° C., quenched with NH₄Cl (20 mL), diluted with EtOAc (200 mL), washed with NH₄Cl (2×50 mL), NaHCO₃ (50 ml), brine (50 mL), dried over MgSO₄ and concentrated. Chromatography on silica with 8% EtOAc in hexane, then 12% EtOAc in hexane, gave 3.88 g (86%) of ketone 14 as a yellowish oil. ¹H NMR δ 7.34-7.22 (m, 5H), 6.79 (m, 1H), 5.57 (d, J=10.5, 1H), 5.00 (m, 1H), 4.54 (d, J=12.4, 1H), 4.43 (d, J=12.0, 1H), 3.71 (d, J=4.4, 2H), 2.62 (m, 1H), 2.54 (m, 3H), 2.00-1.82 (m, 2H), 1.44 (s, 9H). ¹³C NMR δ 195.0, 155.5, 145.5, 143.3, 137.7, 128.4, 127.8, 127.6, 79.8, 73.2, 71.1, 56.4, 34.3, 31.0, 28.4, 22.5. Anal. Calcd. for: C₂₀H₂₇O₄N: C, 69.54; H, 7.88; N, 4.05. Found: C, 69.54; H, 7.74; N, 4.01.

Alcohol (15). Ketone 14 (3.78 g, 11.0 mmol) was dissolved in 2.5:1 THF/MeOH (125 ml) and cooled to 0° C. CeCl₃ (4.91 g, 13.2 mmol) was added, followed by NaBH₄ (0.84 g, 22 mmol). After stirring 2 h at 0° C., the reaction was quenched with NH₄Cl (50 mL), diluted with EtOAc (200 mL), washed with NH₄Cl (2×100 mL), brine (100 mL), dried on MgSO₄ and concentrated. Chromatography on silica with 15% EtOAc in hexane yielded 3.49 g (92%) of a white solid as a 4:1 mixture of diastereomers. m.p. 67-68° C. The major diastereomer was isolated by precipitation from EtOAc/n-hexane. ¹H NMR δ 7.36-7.28 (m, 5H), 5.65 (m, 1H), 5.35 (d, J=8.4, 1H), 4.51 (d, J=11.6, 1H), 4.42 (d, J=12.0, 1H), 4.33 (br, s, 1H), 3.84 (br, s, 1H), 3.71-3.68 (dd, J=3.4, 13.4, 1H), 3.60-3.55 (dd, J=2.6, 9.4, 1H), 3.18 (d, J=8.4, 1H), 2.35-2.20 (m, 4H), 1.87 (m, 2H), 1.44 (s, 9H) ¹³C NMR δ 155.9, 144.7, 137.6, 128.7, 128.2, 128.1, 126.7, 79.7, 74.1, 74.0, 70.6, 52.1, 32.4, 28.6, 23.9. Anal. Calcd for: C₂₀H₂₉O₄N: C, 69.14; H, 8.41; N, 4.03. Found: C, 69.42; H, 8.54; N, 4.12.

Ester (16). To a solution of alcohol 15 (3.26 g, 9.38 mmol) and pyridine (2.28 mL, 28.2 mmol) in THF (4 mL) was added a solution of t-butyldimethylsilyloxyacetyl chloride (2.05 g, 9.40 mmol) in THF (4 mL) dropwise at 0° C. The reaction was stirred for 3 h at rt then diluted with 30 mL Et₂O, washed with 0.5 N HCl (2×20 mL), NaHCO₃ (10 mL), brine (10 mL), dried on MgSO₄ and concentrated. Chromatography with 4% EtOAc in hexanes on silica gave 3.48 g (70%) of ester 16 as a yellow oil. ¹H NMR δ 7.35-7.28 (m, 5H), 5.67 (s, 1H), 5.58 (d, J=8.0, 1H), 4.83 (d, J=9.4, 1H), 4.51 (d, J=11.9, 1H), 4.42 (d, J=11.9, 1H), 4.16 (s, 2H), 4.04 (m, 1H), 3.55 (dd, J=3.5, 9.4, 1H), 3.48 (dd, J=3.3, 9.5, 1H), 2.41 (m, 1H), 2.33-2.21 (m, 3H), 1.83 (m, 2H), 1.40 (s, 9H), 0.90 (s, 9H), 0.07 (s, 6H). ¹³C NMR δ 170.6, 155.3, 139.9, 138.0, 130.2, 128.5, 127.8, 127.7, 79.5, 73.3, 72.6, 68.5, 61.8, 51.0, 32.4, 31.6, 28.4, 25.8, 23.2, 18.4, −5.4. Anal. Calcd for: C₂H₄₅NO₄Si: C, 64.70; H, 8.73; N, 2.69. Found: C, 64.58; H, 8.89; N, 2.69.

α-Hydroxy acid (17). To a solution of diisopropylamine (3.3 mL, 24 mmol) in THF (40 mL) was added n-butyl lithium (2.5 M in hexane, 8.6 mL, 22 mmol) at 0° C. The mixture was stirred for 15 min to generate LDA. Then a mixture of chlorotrimethyl silane (7.52 mL, 59.2 mmol) and pyridine (5.22 mL, 64.6 mmol) in THF (15 mL) was added dropwise to the LDA solution at −100° C. After 5 min, a solution of ester 16 (2.83 g, 5.38 mmol) in THF (18 mL) was added dropwise and the reaction was stirred at −100° C. for 25 min then warmed slowly to rt over 1.5 h and stirred at rt for 1.5 h. The reaction was quenched with 1 N HCl (70 mL) and the aqueous layer was extracted with Et₂O (2×150 mL). The organic layer was dried on MgSO₄ and concentrated to give 1.98 g (crude yield 70%) colorless glassy oil. Without further purification, the product was dissolved in 10 mL THF. Tetrabutylammonium fluoride (2.8 g, 11 mmol) in THF (10 mL) was added at 0° C., stirred at 0° C. for 5 min then at rt for 1 h. The reaction was quenched with 0.5 N HCl (50 mL), extracted with EtOAc (100 mL), dried on MgSO₄ and concentrated. Chromatography with 50% EtOAc in hexane on silica gave 1.16 g (52%) of α-hydroxy acid 17 as a colorless foam. ¹H NMR (DMSO-d₆) δ 7.36-7.24 (m, 5H), 6.84 (d, J=7.35, 1H), 5.28 (d, J=7.80, 1H), 4.50 (d, J=11.9, 1H), 4.44 (d, J=12.2, 1H), 4.31 (br, s, 1H), 3.84 (d, J=6.0, 1H), 3.40-3.32 (m, 2H), 3.27 (dd, J=5.1, 10.1, 1H), 2.70-2.61 (m, 1H), 2.41-2.37 (m, 1H), 2.17-2.10 (m, 1H), 1.74-1.67 (m, 2H), 1.55-1.42 (m, 2H), 1.37 (s, 9H). ¹³C NMR (DMSO-d₆) δ 175.3, 155.7, 145.4, 139.2, 128.7, 127.9, 127.8, 121.6, 78.0, 74.0, 72.5, 72.3, 50.5, 47.6, 30.0, 29.6, 28.8, 24.6. Anal. Calcd for: C₂₂H₃₁NO₆: C, 65.17; H, 7.71; N, 3.45. Found: C, 65.03; H, 7.80; N, 3.47.

Acid (18). Lead tetraacetate (2.69 g, 6.06 mmol) in CHCl₃ (13.5 mL) was added dropwise to a solution of acid 17 (2.28 g, 5.51 mmol) in EtOAc (81 mL) at 0° C. The reaction was stirred for 10 min then quenched with ethylene glycol (8 mL), diluted with EtOAc (150 mL), washed with H₂O (4×15 mL), brine (15 mL), dried on Na₂SO₄ and concentrated to give 2.02 g (100% crude yield) aldehyde as yellowish oil. ¹H NMR(CHCl₃) δ 9.38 (d, J=2.8, 1H), 7.36-7.27 (m, 5H), 5.39 (dd, J=2.2, 8.6, 1H), 4.95 (d, J=7.1, 1H), 4.55 (d, J=12.2, 1H), 4.47 (d, J=12.2, 1H), 4.41 (br, s, 1H), 3.50 (dd, J=4.3, 9.3, 1H), 3.43 (dd, J=5.0, 9.4, 1H), 3.25 (m, 1H), 2.55 (m, 1H), 2.24 (m, 1H), 1.99 (m, 1H), 1.86 (m, 1H), 1.72 (m, 2H), 1.43 (s, 9H). The product was dissolved in acetone (140 mL) and cooled to 0° C. Jones reagent (2.7 M H₂SO₄, 2.7 M CrO₃, 4 mL, 11 mmol) was added dropwise. The reaction was stirred at 0° C. for 0.5 h and quenched with isopropyl alcohol (12 mL) and stirred for 10 min. The precipitate was filtered out and the solvent was evaporated. The residue was extracted with EtOAc (3×200 mL), washed H₂O (50 mL), brine (50 mL), dried on Na₂SO₄ and concentrated. Chromatography on silica with 30% EtOAc in hexane gave 1.65 g (78%) of acid 18 as a colorless oil. ¹H NMR (CHCl₃) δ 7.30 (m, 5H), 5.55 (d, J=6.7, 1H), 4.93 (br, s, 1H), 4.53 (d, J=12.1, 1H), 4.51 (d, J=12.1, 1H), 4.39 (br, s, 1H), 3.47 (dd, J=3.5, 9.2, 1H), 3.41 (dd, J=5.3, 9.6, 1H), 3.36 (t, J=7.0, 1H), 2.54 (m, 1H), 2.29 (m, 1H), 2.04-1.84 (m, 3H), 1.66 (m, 1H), 1.43 (s, 9H). ¹³C NMR (CHCl₃) δ 179.9, 155.6, 143.8, 138.2, 128.5, 127.7, 127.6, 122.6, 79.4, 73.1, 72.1, 50.4, 49.5, 30.1, 29.4, 28.5, 25.1. IR (cm⁻¹): 3000-2800 (br), 1701 (s), 1162, 731, 697. HRMS calcd for C₂₁H₂₉NO₅ (MH⁺) m/z=376.2124, found m/z=376.2133.

Boc-Ser-Ψ[(E)CH═C]Pro-OH (2). NH₃ (35 mL) was distilled, allowed to warm to reflux (−33° C.) and Na (ca. 330 mg, 14 mmol) was added until a deep blue solution was sustained. Acid 18 (575 mg, 1.50 mmol) in THF (13 mL) was added directly to the Na/NH₃ solution via syringe. After stirring 15 min at reflux, the reaction was quenched with NH₄Cl (20 mL), then allowed to warm to rt. NH₄Cl (40 mL) was added, and the mixture was extracted with CHCl₃ (5×30 mL). The aqueous layer was acidified with 1 N HCl and extracted with CHCl₃ (6×50 mL). The CHCl₃ layer was dried on MgSO₄ and concentrated to give 280 mg (64%) of the acid as a yellowish oil. ¹H NMR (DMSO-d₆) δ 6.66 (d, J=7.4, 1H), 5.31 (dd, J=2.1, 8.7, 1H), 4.61 (br, s, 1H), 4.06 (s, 1H), 3.27 (dd, J=7.1, 10.8, 1H), 3.20 (dd, J=5.7, 10.5, 1H), 3.16 (m, 1H), 2.39 (m, 1H), 2.22 (m, 1H), 1.80 (m, 3H), 1.52 (m, 1H), 1.36 (s, 9H). ¹³C NMR δ 175.4, 155.7, 143.6, 122.5, 78.0, 64.0, 52.9, 49.6, 30.1, 29.5, 28.8, 25.0. HRMS calcd for C₁₄H₂₃NO₅ (MH⁺) m/z=286.1654, found m/z=286.1661.

### Example 5

Ac-Phe-Tyr-phosphoSer-Ψ[CH═C]-Pro-Arg-NH₂ has been made and tested as follows:

Previously we have described the syntheses of an exactly matched pair of conformationally locked peptidomimetics as Pin1 inhibitors and cancer cell anti-proliferative reagents, based on the Pin1 preference for aromatic residues N-terminal to the central Ser and an Arg residue C-terminal to the central Pro. (Wang et al. 2004, supra) We have now synthesized 28 mg of Ac-Phe-Tyr-pSerΨ[(Z)CH═C]-Pro-Arg-NH₂ 21.

Boc-Ser-Ψ[(Z)CH═C]-Pro-OH 1 and Boc-Ser-Ψ[(E)CH═C]-Pro-OH 2, were reprotected as the Fmoc-carbamates 19 and 20 (Scheme 4). Deprotections of Boc by acidolysis were carried out in the presence of triethylsilane as a carbocation scavenger, greatly improving the yields. Reactions with Fmoc-Cl were conducted by adding saturated Na₂CO₃ intermittently to maintain the pH between 8 and 9, giving the Fmoc-protected compounds 19 with a two-step yield of 68%.

Phosphorylation via a building block approach was found to give the best results in each case, although global phosphorylation to give the N-methylcarboxamide was also successful. The unsymmetrical phosphoramidite, O-benzyl-O-β-cyanoethyl-N,N-diisopropylphosphoramidite, was originally used as a phosphorylation reagent for the synthesis of a glycolipid. The β-cyanoethyl group can be removed by piperidine simultaneously with Fmoc deprotection to leave the phosphate mono-anion, which is the most stable form of phosphoserine in peptide synthesis. The dipeptide analogues were phosphorylated in a one-pot reaction according to published procedures with minor modifications. Each Fmoc-protected isostere was treated with one equivalent each of TBSCl and NMM, which selectively blocked the carboxyl group and left the side chain hydroxyl group free. Phosphitylation with 5-ethylthio-1H-tetrazole, followed by oxidation with tert-butyl hydroperoxide, gave the protected phosphodipeptide isostere 19 in 68% yield (Scheme 4). No isomerization of the β,γ-unsaturated acids to the more stable α,β-unsaturated acids occurred during these reactions. We attribute this to the carboxylate anion inhibiting deprotonation at the α-carbon.

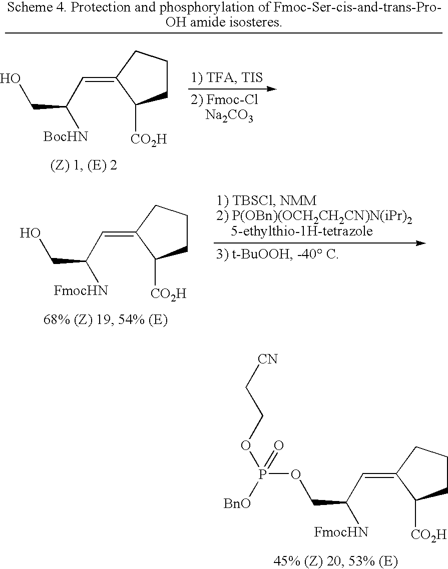

The sequence of this peptidomimetic was slightly modified from our previously synthesized Ac-Phe-Phe-pSerΨ[(Z)CH═C]-Pro-Arg-NH₂ to Ac-Phe-Tyr-pSer-Ψ[(Z)CH═C]-Pro-Arg-NH₂ 21. A tyrosine residue replaced the phenylalanine residue to closely resemble the best substrate for Pin1, Ac-Trp-Phe-Tyr-pSer-Pro-Arg-pNA.

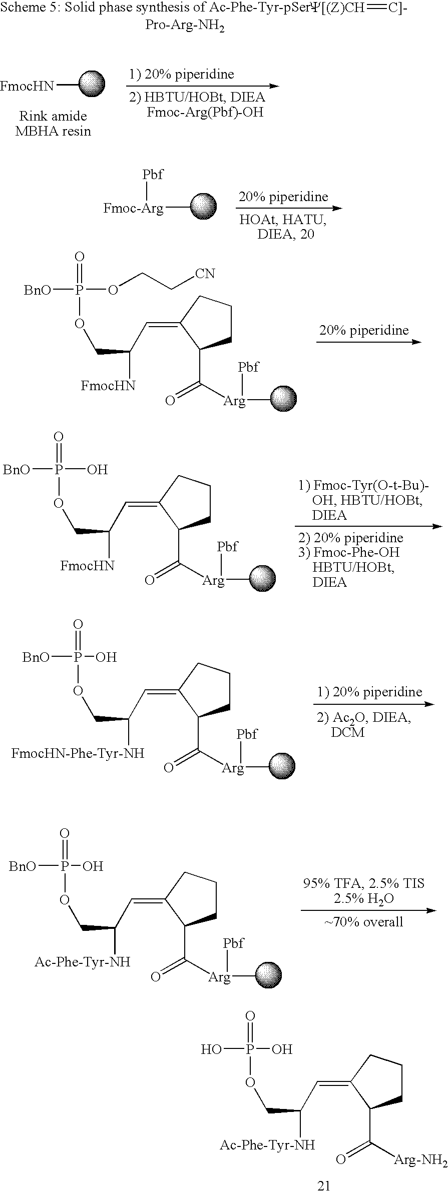

The peptidomimetic 21 was synthesized using Rink MBHA resin. The standard Fmoc solid phase peptide synthesis was applied with modification. Boc protection on tyrosine hydroxyl group was used as recommended in SPPS methods (Novabiochem catalogue, 2004, Synthesis Notes, S1-S96.). A coupling time of 20 minutes for each amino acid was used. Double coupling was conducted if a Kaiser test indicated the first coupling was not quantitative. Coupling reagents HOAt and HATU were used for coupling of the dipeptide building block, at a coupling time of 90 minutes. The (Z)-alkene dipeptide isostere building block isomerized under amino acid coupling condition but at a rate slower than its (E)-alkene counterpart. After the coupling of the dipeptide building block onto the resin, the resin was exposed to 20% piperidine for 20 minutes total for the Fmoc deprotection. Acetic acid washing to remove residual NMP and drying over KOH for overnight were performed right before the cleavage of the peptide from the resin. The peptide was cleavage with 95% TFA, 2.5%, and 2.5% water.

After HPLC purification by a semi-prep C₁₈ column, the peptidomimetic 21 was obtained as a white solid in ca. 72% yield, which is a typical yield for solid phase peptide synthesis. It was characterized by MS-ESI, ¹H NMR, ³¹P NMR and ¹³C NMR. Notably, although the peptidomimetic Ac-Phe-Tyr-pSerΨ[(Z)CH═C]-Pro-Arg-NH₂ and previously synthesized Ac-Phe-Phe-pSerΨ[(Z)CH═C]-Pro-Arg-NH₂ vary by only a hydroxyl group on the benzene ring, the coupling patterns in the ¹H NMR spectra are very different.

The yield of the peptidomimetics Ac-Phe-Phe-pSerΨ[(Z)CH═C]-Pro-Arg-NH₂ and Ac-Phe-Phe-pSerΨ[(E)CH═C]-Pro-Arg-NH₂ were improved to 46% and 42%, respectively, using the method of this Example.

Fmoc-SerΨ[(Z)CH═C]-Pro-OH (19) Boc-SerΨ[(Z)CH═C]-Pro-OH 1 (114 mg, 0.40 mmol) was dissolved in a solution of 1:3 TFA:CH₂Cl₂ (10 mL) at 0° C. The reaction mixture was stirred for 45 min at room temperature and the solvent was evaporated. The remaining TFA was removed under vacuum at room temperature. Without further purification, the crude product was dissolved in a 10% Na₂CO₃ aq (2.0 mL), then cooled to 0° C. A solution of Fmoc-Cl (114 mg, 0.44 mmol) in dioxane (2.0 mL) was added slowly to the above reaction mixture and stirred at room temperature for 3 h. The reaction mixture was diluted with H₂O (30 mL) and extracted with ether (2×20 mL). The aqueous layer was acidified with 1N HCl to pH 3, and then extracted with EtOAc (3×30 mL) and CH₂Cl₂ (3×30 mL). The combined organic layer was dried over MgSO₄ and concentrated to give 120 mg of the crude product.

Chromatography with 0.5% acetic acid and 5% MeOH in CH₂Cl₂ eluted 94 mg (58% yield) of the product as a white solid. ¹H NMR (DMSO-d₆) δ 12.1 (br s, 1H), 7.87 (d, J=7.6, 2H), 7.71 (d, J=7.6, 2H), 7.40 (app. t, J=7.4, 2H), 7.32 (app. t, J=7.4, 2H), 7.12 (d, J=7.6, 1H), 5.31 (d, J=9.2, 1H), 4.65 (br s, 1H), 4.24-4.17 (m, 4H), 3.44 (m, 1H), 3.38 (dd, J=10.6, 5.4, 1H), 3.24 (m, 1H), 2.31 (m, 1H), 2.22 (m, 1H), 1.88 (m, 2H), 1.74 (m, 1H), 1.53 (m, 1H). ¹³C NMR (DMSO-d₆) δ 175.2, 155.3, 144.0, 143.9, 143.0, 140.7, 127.6, 127.0, 125.3, 122.2, 120.0, 65.3, 63.7, 52.4, 46.7, 45.4, 33.4, 31.1, 24.1.

Fmoc-Ser(PO(OBn)(OCH₂CH₂CN))-Ψ[(Z)CH═C]-Pro-OH (20). NMM (25.3 mg, 0.25 mmol) was added to a stirred solution of Fmoc-SerΨ[(Z)CH═C]-Pro-OH 19 (100 mg, 0.25 mmol) in THF (2 mL), followed by t-butyldimethylsilyl chloride (TBSCl, 41.5 mg, 0.27 mmol). After 30 min, a solution of O-benzyl-O-β-cyanoethyl-N,N-diisopropylphosphoramidite (154 mg, 0.5 mmol) in THF (1 mL) was added, followed by 5-ethylthio-1H-tetrazole (130 mg, 1.0 mmol) in one portion. The reaction mixture was stirred for 3 h at room temperature, then cooled to −40° C. and tert-butyl hydroperoxide (5 M in decane, 100 μl, 0.5 mmol) was added. The cold bath was removed. After stirring for 30 min, the mixture was again cooled to −40° C., and 4 mL of 10% aq. Na₂S₂O₃ was added. The solution was extracted using ether (2×40 mL). The organic layer was dried over MgSO₄ and concentrated. Chromatography on silica gel with 10% acetone in CH₂Cl₂ to remove impurities, then 1% acetic acid and 10% acetone in CH₂Cl₂ eluted 96 mg (62%) of 20 as a colorless syrup. ¹H NMR (DMSO-d₆) δ 12.19 (br, s, 1H), 7.90 (d, J=7.6, 2H), 7.69 (d, J=7.2, 2H), 7.50 (d, J=7.6, 1H), 7.43-7.29 (m, 9H), 5.37 (d, J=8.4, 1H), 5.04 (dd, J=3.8, 7.8, 2H), 4.50 (m, 1H), 4.29-4.20 (m, 3H), 4.13 (m, 2H), 3.96-3.87 (m, 2H), 3.43 (t, J=6.0, 1H), 2.88 (m, 2H), 2.31 (m, 1H), 2.24 (m, 1H), 1.87 (m, 3H), 1.73 (m, 1H). ¹³C NMR (DMSO-d₆) δ 174.9, 155.3, 145.1, 143.9 (d, J_{PC}=8.3), 140.7, 135.8 (dd, J_{PC}=2.3, 6.8), 128.5, 128.4, 127.8 (d, J_{PC}=3.1), 127.6, 127.1, 125.2 (d, J_{PC}=3.8), 120.1, 119.6, 118.2 (d, J_{PC}=1.5), 68.7 (d, J_{PC}=5.3), 68.3, 65.5, 62.3 (dd, J_{PC}=2.3, 5.3), 50.2 (d, J_{PC}=8.5), 46.6, 45.5, 33.5, 31.0, 24.1, 19.0 (d, J_{PC}=7.6). ³¹P NMR (DMSO-d₆) δ −1.762.

Ac-Phe-Tyr-pSerΨ[(Z)CH═C]-Pro-Arg-NH₂ (21). The solid phase synthesis of Ac-Phe-Tyr-pSerΨ[(E)CH═C]-Pro-Arg-NH₂, 21, was performed manually by standard Fmoc chemistry. Rink amide MBHA resin (156 mg, 0.10 mmol, loading: 0.64 mmol g⁻¹) was swelled in CH₂Cl₂ (3 mL, 10 min) and then N-methylpyrrolidinone (NMP) (3 mL, 10 min). Amino acids (Arg, Tyr, and Phe) were either coupled once (Tyr) or double coupled (Arg and Phe). In each cycle, the N-protecting Fmoc group was removed by 20% piperidine in NMP (2×3 mL, 10 min each). After washing with NMP (5×3 mL) and DCM (5×3 mL, a solution of amino acid (Arg, Tyr, and Phe, 0.30 mmol, 3 eq), HBTU (114 mg, 0.30 mmol, 3 eq), HOBt (46 mg, 0.30 mmol, 3 eq) and DIEA (78 mg, 0.6 mmol, 6 eq) was added to the resin and shaken for 20 min. Double coupling was conducted if Kaiser test indicated the coupling was not quantitative. For the coupling of the dipeptide isostere, Fmoc-Ser (PO(OBn)(OCH₂CH₂CN))Ψ[(Z)CH═C]-Pro-OH, 20, HATU, HOAt and DIEA were added to resin, followed by a solution of 20 in 3 mL NMP, the reaction was shaken for 90 min, washed with NMP (5×3 mL) and DCM (5×3 mL), and then capped with 10% Ac₂O, 10% DIEA in CH₂Cl₂ (3 mL) for 15 min. The cyanoethyl group was removed with 20% piperidine in NMP simultaneously with Fmoc (2×3 mL, 10 min each). Final acetylation was carried out with 10% Ac₂O, 10% DIEA in CH₂Cl₂ (4 mL) for 30 min. Then the resin was washed with DCM (5×4 mL), acetic acid (5×4 mL), MeOH (5×4 mL), and ether (3×4 mL) and dried in vacuo over KOH overnight.

The dried resin was treated with a mixture of 95% TFA, 2.5% H₂O, 2.5% TIS (4 mL) for 4 h, filtered and rinsed with TFA. The combined solution was concentrated to a small volume. The crude product was triturated with ether and dried in vacuo to give 80 mg of crude product.

A 40 mg fraction of the crude product was purified by preparative HPLC on a 100×212 mm Varian Polaris C₁₈ column (10μ□). 20 mg (yield 72%) of the product was eluted as a white solid. ¹H NMR (DMSO-d₆): δ 9.19 (br, s, 1H), 8.14 (d, J=8.0, 1H), 7.97 (d, J=7.4, 1H), 7.94 (d, J=7.4, 1H), 7.87 (d, J=8.3, 1H), 7.57 (br, s, 1H), 7.40-6.80 (m, 13H), 6.65 (d, J=8.5, 2H), 5.23 (d, J=7.6, 1H), 4.54 (m, 1H), 4.41 (m, 1H), 4.34 (m, 1H), 4.19 (dd, J=7.8, 13.3, 1H), 3.83 (m, 1H), 3.67 (m, 1H), 3.51 (t, J=6.0, 1H), 3.11 (m, 1H), 2.89 (m, 1H), 2.67 (m, 1H), 2.34 (m, 1H), 2.22 (m, 1H), 1.85 (m, 2H), 1.72 (m, 6H), 1.63 (m, 1H), 1.49 (m, 3H). ¹³C NMR (DMSO-d₆): δ 173.6, 172.8, 171.1, 170.7, 169.6, 156.7, 155.8, 145.3, 138.0, 130.1, 129.0, 128.0, 127.7, 126.1, 120.2, 114.9, 66.4, 54.4, 54.0, 52.1, 49.2, 46.3, 40.5, 36.8, 33.9, 31.6, 28.8, 24.9, 24.1, 22.4. ³¹P NMR (DMSO-d₆): δ −1.012. MS-ESI(+) calcd for C₃₅H₅₀N₈O₁₀P (MH⁺) m/z=773.3, found m/z=773.6.

Measurement of human Pin1 peptidyl-prolyl isomerase activity. The concentration of the cis conformation of substrate, SucAEPF-pNA, was determined by the UV absorbance of pNA (ε=12250 at 390 nM) after cleavage by α-chymotrypsin. The cis component of the substrate was approximately 51%. The assay buffer (1050 μL of 35 mM HEPES, pH 7.8 at 0° C.; final concentration 31 mM HEPES) and Pin1 (10 μL of a 8.0 μM stock solution, concentration measured by Bradford assay, final concentration 67 nM) were pre-equilibrated in the spectrometer until the temperature reached 4.0° C. Immediately before the assay was started, 120 μL of ice-cooled α-chymotrypsin solution (60 mg/mL in 0.001 M HCl; final concentration 6 mg/mL) was added. Additional substrate solvent (0.47 M LiCl/TFE) was added as needed to bring the total volume of substrate and cosolvent to 10 μL. The peptide substrate, dissolved in dry 0.47 M LiCl/TFE, was added to the cuvette via syringe and the solution was mixed vigorously by inversion 3 times. The final volume in a semi micro 1 cm path length polystyrene cell was 1.2 mL.

IC₅₀ measurement of inhibitors for Pin1. Human Pin1 was assayed at a cis substrate concentration of 43.2 μM. The assay buffer (1050 μL of 35 mM HEPES, pH 7.8; final concentration 31 mM HEPES), Pin1 (10 μL of stock solution) and inhibitors (10 μL of varying concentration in 1:3 DMSO:H₂O) were preequilibrated in the cuvette at 4° C. for 10 min. Immediately before the assay was started, 120 μL of ice-cooled chymotrypsin solution (60 mg/mL in 0.001 M HCl; final concentration 6 mg/mL) was added. Peptide substrate SucAEPF-pNA (10 μL), dissolved in 0.47 M LiCl/TFE was added to the cuvette and the solution was mixed vigorously. After a mixing delay of 6-8 sec, the progress of the reaction was monitored by absorbance at 390 nM for 90 sec.

### Example 6

Phospho-(D) serine analogues are made as follows:

Still-Wittig Route to (D)-Ser-cis-Pro Mimic. The key steps in the synthesis of Boc-(D)-Ser-Ψ[(Z)CH═C]-Pro-OH are stereoselective reduction of the ketone to the (R,S)-alcohol, and Still-Wittig rearrangement to the (Z)-alkene. Starting with the Weinreb amide of Boc-(D)-Ser(OBn)-OH, the reaction of the Weinreb amide with cyclopentenyl lithium gives the desired ketone. The reaction is difficult to bring to completion, even with excess cyclopentenyl lithium, probably due to deprotonation of the carbamate. The yield is increased by adding three equivalents of cyclopentenyl lithium in portions. The chelation-controlled Luche reduction of the ketone gives the desired diastereomer as the major product. The iodomethyltributyl tin reagent was prepared by the method of Steitz et al. Fractional distillation is recommended. After forming the intermediate tributylstannylmethyl ether, Still-Wittig rearrangement gives a mixture of alkenes with the (Z)-alkene in excess. The diastereomers can be separated by column chromatography.

Taking the (Z)-alkene, the benzyl side chain and Boc-amine protections are removed and the amine is reprotected for peptide synthesis. The benzyl of the side chain is removed by sodium/ammonia reduction. Jones oxidation on the Boc-amine produces the acid, Boc-(D)-Ser-Ψ[(Z)CH═C]Pro-OH.

Phosphorylation and incorporation of this dipeptide analogue into peptidomimetics is performed in a manner directly analogous to the natural (L)-Ser isostere.

### Example 7

Fmoc-bis(pivaloylmethoxy) phosphoSer-Ψ[CH═C]-Pro-2-aminoethyl-(3-indole) is made as follows.

Referring to the above results in Examples 1-6, the inventors considered design of more potent inhibitors than the cis isotere by introducing the bis-pivaloyloxymethyl(POM) phosphate trimesters. In order to achieve this, two target molecules were designed:

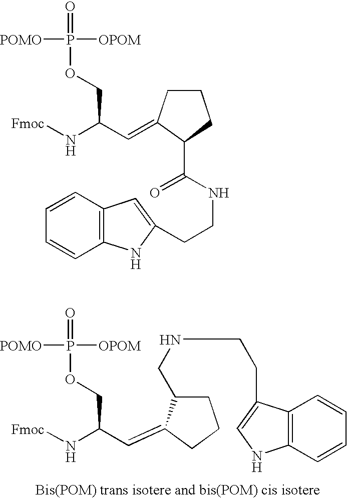

The bis(POM) phosphate is introduced into cis or trans isoteres, and then coupled with tryptamine.

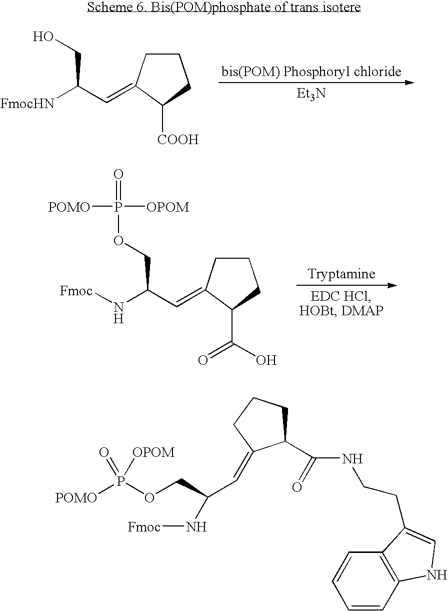

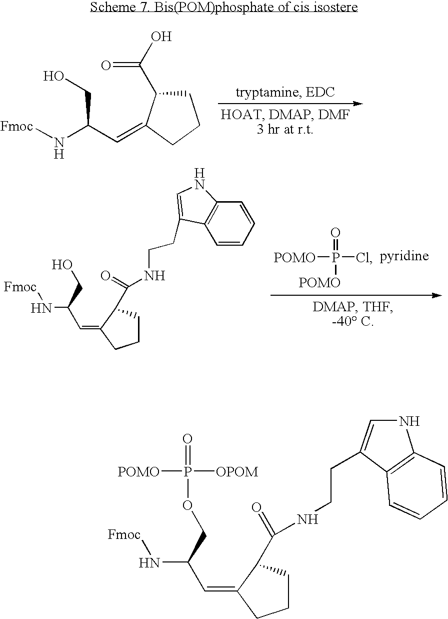

The synthesis of Fmoc-bis(pivaloylmethoxy) phosphoSer-Ψ[CH═C]-Pro-2-aminoethyl-(3-indole) is shown in Scheme 7. Fmoc-Ser-Ψ[(Z)CH═C]-Pro-OH 1 was used for the coupling reaction with tryptamine, then bis(POM)-phosphoryl chloride was used to introduce the bis(POM) phosphate. The coupling reaction between cis mimic and tryptamine was run without adding base. When the reaction for introducing bis(POM)phosphate was run at −40° C. and the weak base pyridine was used, no β-elimination product was observed and the desired product was obtained.

For the synthesis of Bis(POM)-phosphoryl chloride (Scheme 8), generally procedures according to Cole et al. were followed, with modification as follows. In order to increase the yield, anhydrous acetonitrile was used and the reaction was run for longer time to reduce the formation of bis(POM) and one POM phosphate.

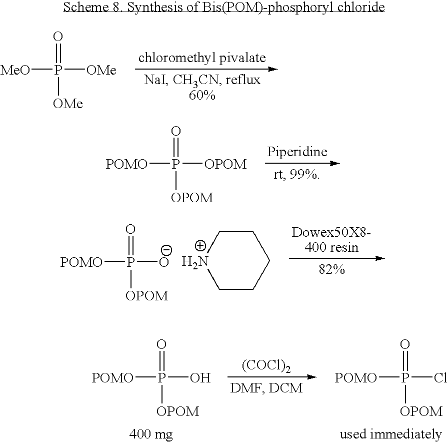

### Experimental Section:

Fmoc-Ser-Ψ[(Z)CH═C]-Pro-2-aminoethyl-(3-indole): 29 mg Fmoc-Ser-Ψ[(Z)CH═C]-Pro-OH mimic 1 (0.098 mmol) was dissolved in dry DMF (3 mL), cool to 0° C., then EDC. HCl (18.8 mg, 0.098 mmol) was added to the solution slowly, followed by HOAT (13.0 mg, 0.098 mmol) and DMAP (3.54 mg, 0.0298 mmol). The solution became yellowish. Finally, tryptamine (15.7 mg, 0.098 mmol) was added to the solution slowly. The resulting solution was allowed to stand at room temperature for 3 hours. Then the mixture was diluted with 30 mL EtOAc. The organic layer was washed with saturated NaHCO₃ (2×10 mL) and H₂O (2×10 mL) and brine (1×10 mL), dried over Na₂SO₄, filtered, and concentrated. The residue was purified by silica chromatography with CHCl₃ and 0.5% MeOH in CHCl₃ to elute the product (9 mg, 25% yield) as a colorless oil. ¹H-NMR (400 MHz, CDCl₃) δ7.81 (s, 1H), δ 7.75 (d, J=7.2 Hz, 2H), δ7.51 (dd, J=7.0, 11.0 Hz, 3H), δ7.38 (t, J=5.0 Hz, 3H), δ7.29 (t, J=7.0 Hz, 2H), δ 7.05 (t, J=5.0 Hz, 1H), δ 6.95 (t, J=5.0 Hz, 1H), δ 5.2 (d, J=2.0 Hz, 1H), δ 5.1 (s, 1H), δ 4.39 (m, 1H), δ 4.25 (m, 1H), δ4.05 (m, 1H), δ3.78 (m, 1H), δ 3.4 (m, 4H), δ 3.2 (m, 1H), δ 2.95 (m, 1H), δ 2.90 (m, 1H), δ 2.35 (m, 1H), δ2.20 (m, 1H), δ 1.95 (m, 3H), δ 1.90 (m, 1H), δ 1.50 (m, 2H).

Fmoc-bis(pivaloylmethoxy)phosphoSer-Ψ[(Z)CH═C]-Pro-2-aminoethyl-(3-indole). Fmoc-Ser-Ψ[(Z)CH═C]-Pro-2-aminoethyl-(3-indole) (10 mg, 0.018 mmol) was dissolved in freshly distilled THF (0.5 mL). The solution was cooled to 40° C. for 10 min, pyridine (0.5 mL) was added slowly to the solution, followed by DMAP (0.5 mg, 0.004 mmol). The solution was kept at −40° C. for another 10 min. Bis(POM)phosphoryl chloride, which was prepared freshly starting from hydrogen bis(POM)phosphate (35 mg, 0.103 mmol) and freshly distilled oxalyl chloride (50 μL, 0.50 mmol), was dissolved in a mixture of THF (0.5 mL) and CH₂Cl₂ (0.5 mL). The solution of bis(POM) phosphoryl chloride was added dropwise to the solution of Fmoc-Ser-Ψ[(Z)CH═C]-Pro-2-aminoethyl-(3-indole) at −40° C. On completion of addition, the reaction mixture was stirred for 3 h at −40° C. and then a second solution of bis(POM) phosphoryl chloride (0.05 mmol) in CH₂Cl₂ (0.5 mL) was slowly added at −40° C. After stirring for a further 4 h at −40° C., water (3 mL) was added and the reaction mixture was stirred for 10 min. Organic solvent was removed with rotary evaporator under vacuum, and the residue was extracted with CHCl₃ (3×20 mL). The organic layers were combined and washed with 5% citric acid (2×10 mL), 5% NaHCO₃ (2×10 mL), and H₂O (2×10 mL), finally brine (1×10 mL). The organic layer was dried with magnesium sulfate and concentrated to give 7 mg crude product as a light yellow oil. TLC analysis of the crude product showed that starting material was gone and there was a major new spot with higher Rf value than starting material using EtOAc:hexanes (5:4) as the developing solution. ¹H-NMR (500 MHz, CDCl₃) δ8.0 (s, 1H), δ 7.95 (s, 1H), δ 7.75 (d, J=7.2 Hz, 2H), δ 7.51 (m, 3H), δ7.38 (m, 3H), δ 7.30 (m, 2H), δ 7.05 (t, J=5.0 Hz, 1H), δ 6.95 (m, 1H), δ5.6 (d, J=12 Hz, 1H), δ 5.2 (d, J=2.0 Hz, 1H), δ 5.1 (s, 11H), δ 4.5-3.8 (m, 6H), δ 3.4 (d, J=10 hz, 2H), δ 3.2 (m, 1H), δ 2.95 (m, 1H), δ 2.90 (m, 1H), δ 2.35 (m, 1H), δ 2.20 (m, 1H), δ 1.95 (m, 3H), δ 1.70-1.50 (m, 4H), δ 1.35 (m, 4H), δ 1.20 (s, 18H). ³¹P-NMR (CDCl₃) δ −3.91 (s). IR: 2959.1, 2920.6, 1718, 1522.1, 1448.7, 1259.3, 1158.9, 1077.8.

While the invention has been described in terms of its preferred embodiments, those skilled in the art will recognize that the invention can be practiced with modification within the spirit and scope of the appended claims.

1. A phospho compound, selected from the group consisting of: Ac-Phe-Tyr-phosphoSer-Ψ[CH═C]-Pro-Arg-NH₂; Fmoc-bis(pivaloylmethoxy)phosphoSer-Ψ[CH═C]-Pro-2-aminoethyl-(3-indole); Phospho-(D)-serine mimic Ac-Phe-Tyr-phospho-(D)-Ser-Ψ[CH═C]-Pro-Arg-NH₂ and Phospho-(D)-serine mimic Fmoc-bis(pivaloylmethoxy)phospho-(D)-Ser-Ψ[CH—C]-Pro-2-aminoethyl-(3-indole); wherein Ψ means a pseudo amide.

2. An alkene compound, wherein the alkene compound is selected from the group consisting of: 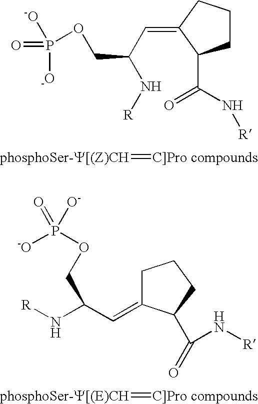
wherein R is a carbonyl group attached to the amine as an amide, and R′ is an amine attached to the carbonyl as an amide.

3. The alkene compound of claim 2, wherein R is selected from the group consisting of the following 26 acid and acid chloride synthons:

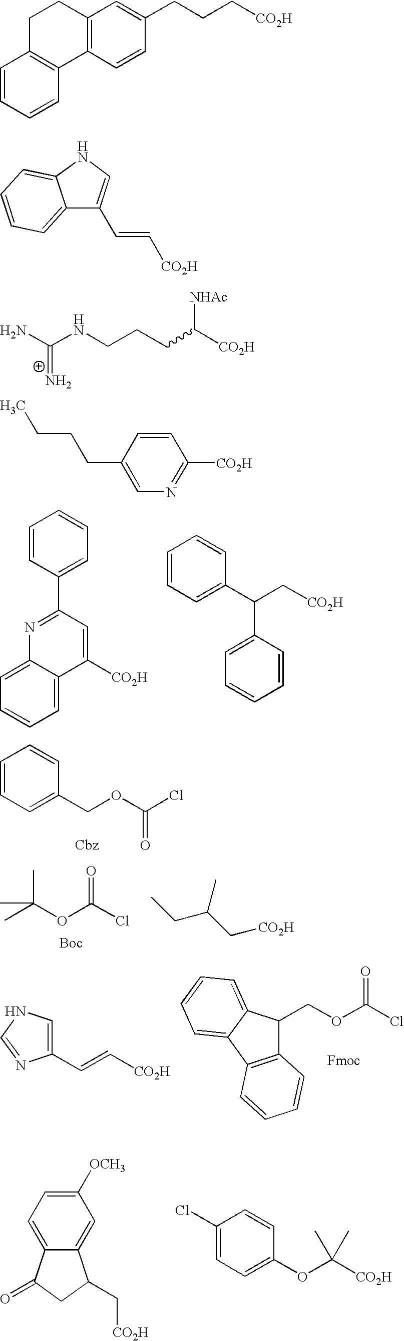

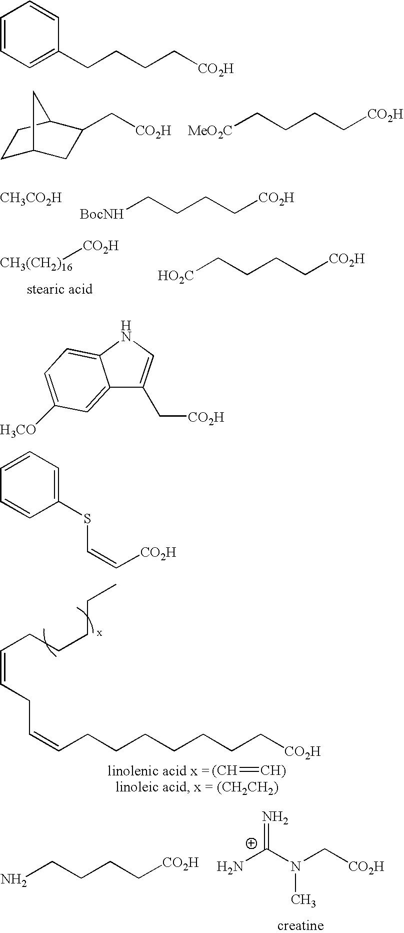

4. The alkene compound of claim 2, wherein R′ is selected from the group consisting of the following 30 amine synthons:

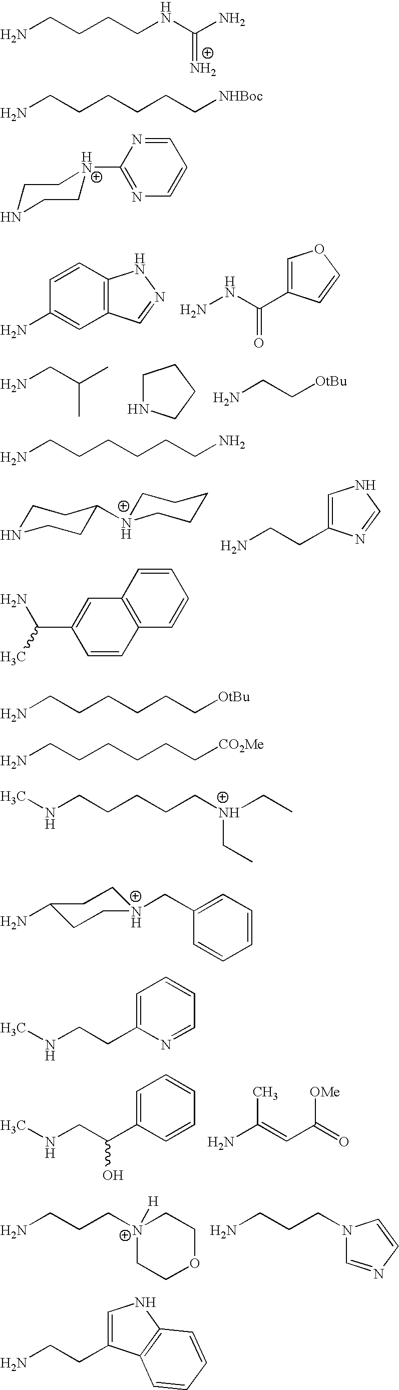

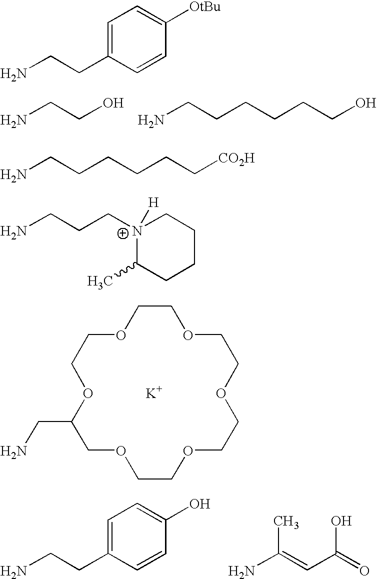

5. The alkene compound of claim 3, wherein R′ is selected from the group consisting of the following 30 amine synthons:

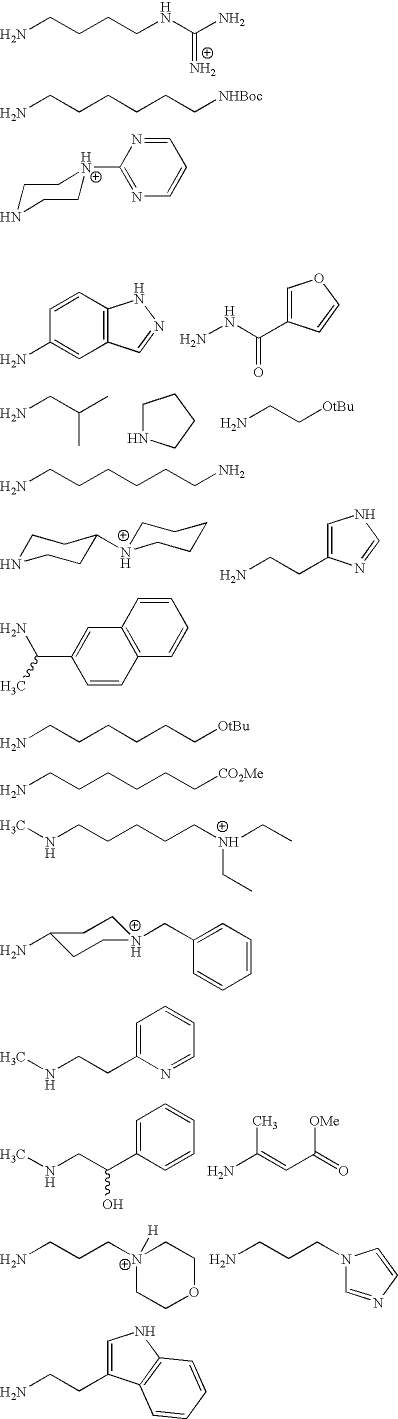

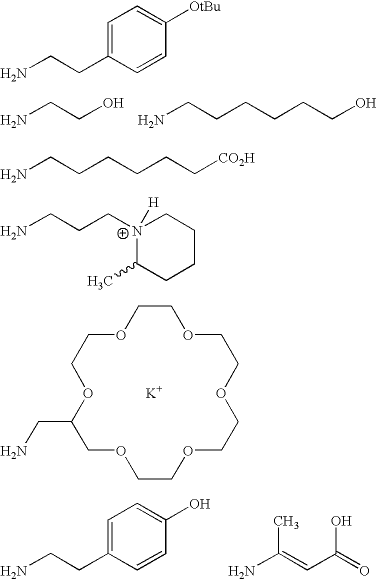

6. The compound of claim 1, which is Ac-Phe-Tyr-phosphoSer-Ψ[(Z)CH═C]-Pro-Arg-NH₂.

7. The compound of claim 1, which is Fmoc-bis(pivaloylmethoxy)phosphoSer-Ψ[(Z)CH═C]-Pro-2-aminoethyl-(3-indole)

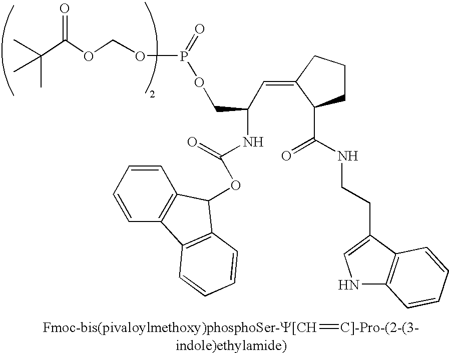

8. An alkene compound which is a phospho-(D)-serine mimic, wherein the phospho-(D)-serine mimic is selected from the group consisting of: 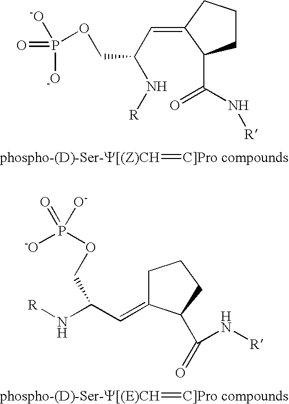
wherein R is a carbonyl group attached to the amine as an amide, and R′ is an amine attached to the carbonyl as an amide.

9. The alkene compound of claim 8, wherein R is selected from the group consisting of the following 26 acid and acid chloride synthons:

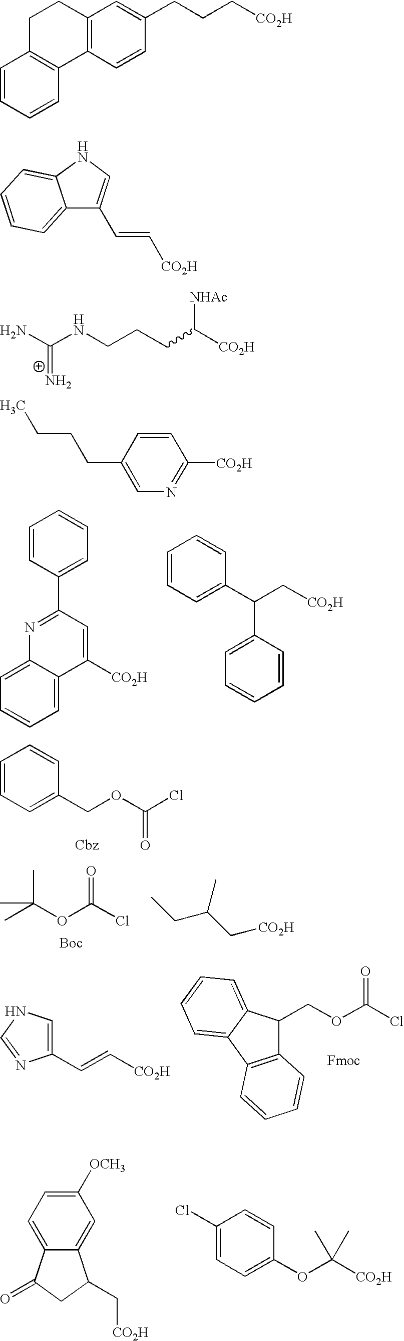

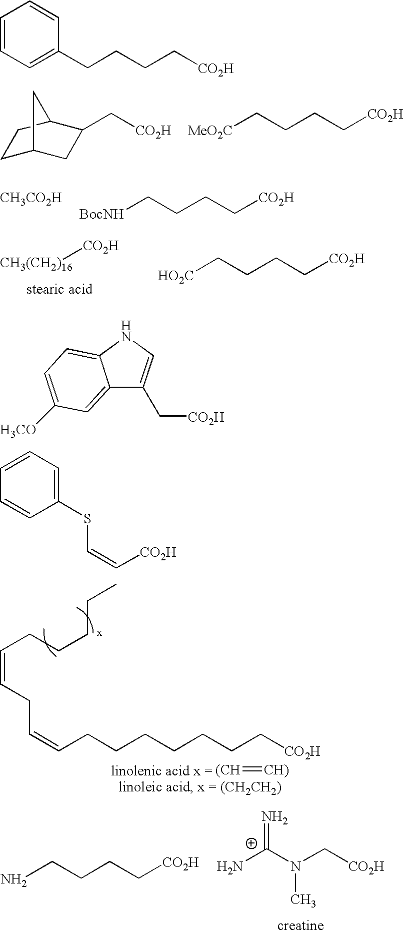

10. The alkene compound of claim 8, wherein R′ is selected from the group consisting of the following 30 amine synthons:

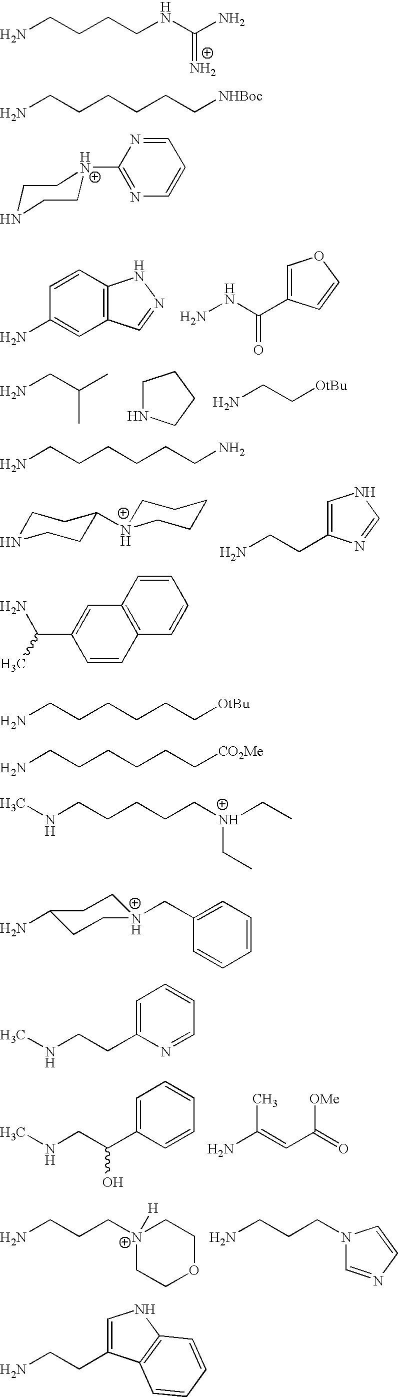

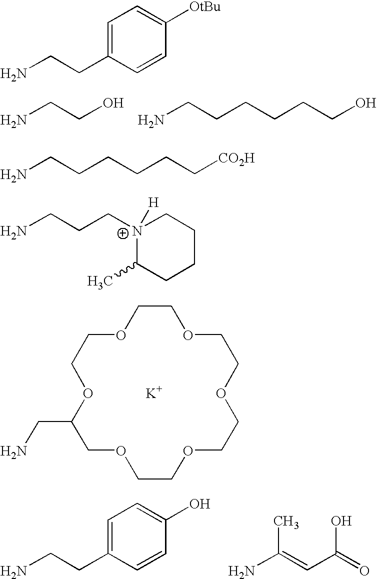

11. The alkene compound of claim 2, wherein the phosphate group is masked as a bis(POM) phosphotriester.

12. The alkene compound of claim 11, wherein the alkene compound is selected from the group consisting of:

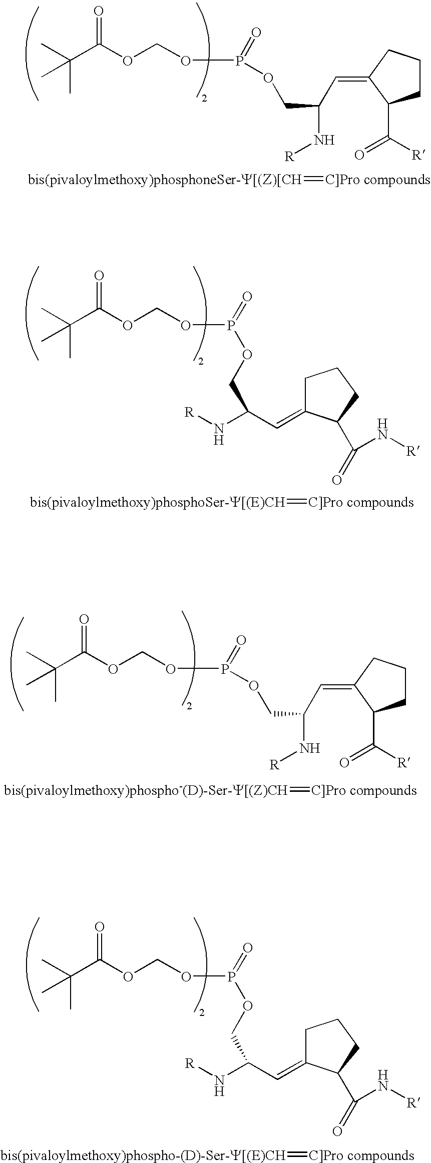

13. A phosphate mimic modified compound comprising the compound of claim 2, modified with at least one phosphate mimic.

14. The phosphate mimic modified compound of claim 13, wherein the phosphate mimic is selected from the group consisting of phosphonate, difluorophosphonate, and bis(pivaloylmethoxy) mimics.

15. The phosphate mimic modified compound of claim 13, wherein the phosphate mimic modified compound is selected from the group consisting of: 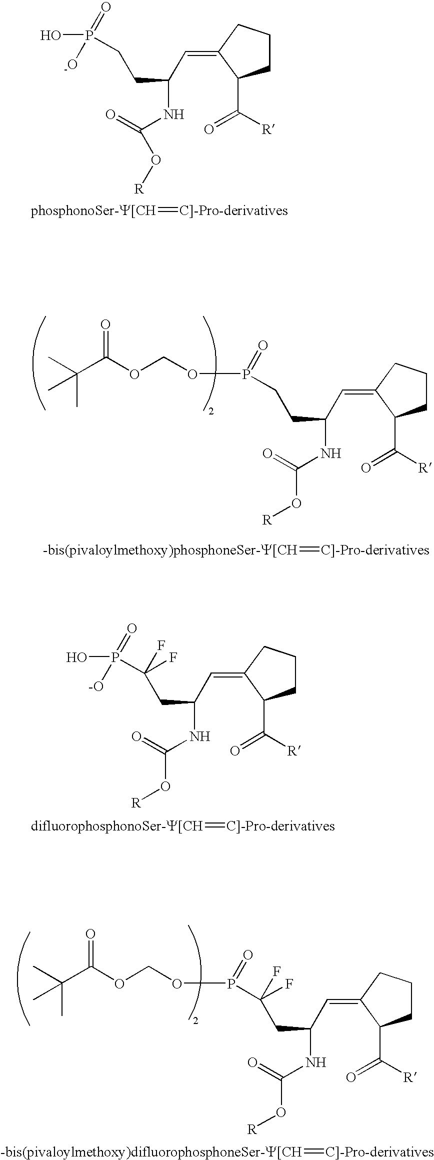
wherein R is a carbonyl group attached to the amine as an amide, and R′ is an amine attached to the carbonyl as an amide.

16. A compound according to claim 1 wherein the compound has (Z) stereochemistry.

17. (canceled)

18. (canceled)

19. (canceled)

20. A method of inhibiting activity against Pin 1, comprising administration of an effective amount of any of the compounds of claim 1, to a subject, wherein Pin 1 activity is inhibited.

21. A method of inhibiting the growth of cancer cells, comprising administration of an effective amount of any of the compounds of claim 1, to a subject having cancer cells, wherein growth of the cancer cells is inhibited.

22. The alkene compounds of claim 10, wherein the phosphate group is masked as a bis(POM) phosphotriester.

23. A method of inhibiting activity against Pin 1, comprising administration of an effective amount of any of the compounds of claim 2, to a subject, wherein Pin 1 activity is inhibited.

24. A method of inhibiting the growth of cancer cells, comprising administration of an effective amount of any of the compounds of claim 2, to a subject having cancer cells, wherein growth of the cancer cells is inhibited.

25. A compound according to claim 2 wherein the compound has (Z) stereochemistry.
